(19) United States
(12) Patent Application Publication
(10) Pub. No.: US 2025/0352799 A1
(43) Pub. Date: Nov. 20, 2025

(54) SYSTEM TO TREAT SLEEP APNEA BY ENTRAINING STIMULATION WITH BREATHING

(71) Applicant: Lunair Medical, Inc., Minneapolis, MN (US)

(72) Inventors: Orhan SOYKAN, Lino Lakes, MN (US); David Wayne BOURN, Champlin, MN (US); Mark GELFAND, New York, NY (US)

(21) Appl. No.: 19/210,987

(22) Filed: May 16, 2025

Related U.S. Application Data
(60) Provisional application No. 63/649,267, filed on May 17, 2024.
(60) Provisional application No. 63/649,240, filed on May 17, 2024.
(60) Provisional application No. 63/649,200, filed on May 17, 2024.

(51) Int. Cl. A61N 1/36; A61F 5/56; A61N 1/02; A61N 1/05; A61N 1/372

(52) U.S. Cl. CPC A61N 1/3611; A61N 1/025; A61N 1/36139; A61N 1/36178; A61N 1/37247; A61F 5/56; A61N 1/0551

(57) ABSTRACT

Techniques for addressing sleep disorders are provided. A system includes a nerve stimulator that is configured to deliver stimulation energy to a nerve of a sleeping patient. A system includes a sensor for gather data from the sleeping patient and a controller for processing the data. The controller is configured to cause the stimulation energy that is provided to be adjusted based on the sensed data.

### CROSS REFERENCE(S) TO RELATED APPLICATION(S)

This application claims priority to U.S. Provisional Application Nos. 63/649,267, 63/649,240, and 63/649,200, all filed on May 17, 2024, the entire contents of each being incorporated by reference herein.

### TECHNICAL FIELD

The invention relates to implantable devices to stimulate phrenic nerves to treat airway collapse in patients with Obstructive Sleep Apnea (OSA). The invention may be embodied to use a pharyngeal mechanoreflex to stiffen the airway, prevent or reverse collapse, improve gas exchange, and/or enhance sleep quality. The invention can be used to keep a sleeping patient comfortable while stimulating the phrenic nerve(s) and/or triggering a reflex to open an obstructed airway in the breathing passage of the patient.

### BACKGROUND

In healthy individuals, airway stability during sleep can be ensured by coordinated and synchronized central control of about 20 (twenty) airway dilator and constrictor muscles (collectively “airway muscles”). The central neural system (CNS) pattern generator (respiratory center) in the medulla of the brain receives inputs from physiologic sensors (also called receptors) via various afferent sensory nerve fibers and controls airway muscles via efferent motor fibers. These physiologic sensors provide physiologic feedback used by the medulla to trigger a reflex in a closed loop reflex arrangement. These reflexes are known as “autonomic” since they do not depend on consciousness.

However, in some instances, the reflexes may become insufficient for optimal health and conditions such as Obstructive Sleep Apnea (OSA) may occur due to, for example, an insufficient reflex response to an obstructed airway.

Sensory inputs to the respiratory center include signals from chemoreceptors that react to oxygen (O2) and carbon dioxide (CO₂) in the arterial blood and many distributed mechanoreceptors including ones that react to transmural pressure across the airway wall. In patients with Central Sleep Apnea (CSA) the former “neurochemical” control loop becomes deranged and may be hyperactive. In patients with snoring and OSA the later “neuromuscular” control loop may become insufficiently active to maintain airway patency.

The airway muscles that keep the upper airway open are accessory muscles of respiration that maintain pharyngeal patency during tidal inspiration. Basal tone in these muscles generally declines at sleep onset. The loss of tone makes the airway prone to collapse and obstruct airflow during sleep.

Afferent receptors in the tracheobronchial tree and lungs detect alterations in airway pressure, temperature, air flow, and lung stretch which may be indicators of a collapsed airway. The afferent receptors provide feedback signals to the CNS which may respond to the feedback signals by triggering reflex responses that stimulate the upper airway muscles, which can then mitigate an airway obstruction.

Over time, in chronic OSA patients, afferent receptors may gradually desensitize and thus the CNS fails to detect the gradual development of airflow obstruction and react to it in time. Under these circumstances, airway neuromuscular activity no longer compensates for the obstruction.

Neuromuscular responses in the upper airway musculature may be coordinated with inspiratory activation of the diaphragm and respiratory pump muscles to maintain patency during sleep.

Neuromodulation therapies can address airway collapsibility by selectively increasing neural signals in the selected efferent branches of the Hypoglossal Nerve (HGN). These branches control protrusion of the tongue by the Genioglossus Muscle (GGM). Also selectively increasing other efferent motor control signals to various dilator muscles, including the ansa cervicalis, can result in in stiffening of the airway.

Increasing lung volume, especially during exhalation, in OSA patients can improve airway patency during sleep. In U.S. Pat. No. 7,970,475 to Tehrani “Device and method for biasing lung volume”, devices and methods are described for increasing lung volume by electrically stimulating of phrenic nerve. Thus, stimulation of phrenic nerve should create mechanical traction on the airway to stiffen it and treat OSA. This approach has limitations since patients can tolerate only modest amounts of additional lung volume without their sleep being disturbed.

Elements of suboptimal anatomy, including chin, neck and tongue anatomy and abdominal obesity, predispose OSA patients to airway collapse. In awake persons, the central neural control compensates for suboptimal anatomy. However, this does not occur during sleep. Artificial Hypoglossal Nerve (HGN) stimulation can address this deficiency, but has limited success. Accordingly, it will be appreciated that new and improved techniques, systems, and processes are continually sought after in this and other areas of technology.

### SUMMARY

In certain example embodiments, a device is used to stimulate peripheral nerves involved in respiration of a patient. This stimulation is provided to leverage existing physiologic autonomic control reflex loops. The techniques described herein may augment and/or restore natural control of the airway stability. The techniques described herein may include: 1) triggering a negative pressure reflex (NPR) in a patient, and 2) triggering direct afferent pathways to the brainstem of a patient.

In certain example embodiments, stimulation therapy (e.g., delivered via an implantable pulse generator) provides stimulation energy to one or more nerves of the patient (e.g., the phrenic nerve, the hypoglossal nerve, etc.) in order to evoke a response of the nerve and result in a therapeutic effect for the patient (e.g., to address sleep apnea). How the stimulation energy is provided may be controlled via stimulation therapy that relies on one or more stimulation parameters. These stimulation parameters may include a stimulation rate, a stimulation phase, a stimulation frequency, a stimulation amplitude, pulse width of stimulation, ramp up for stimulation, plateau time for stimulation, ramp down for stimulation, bi-phasic and mono-phasic stimulation, constant voltage vs constant current. Each of these stimulation parameters may varied according to certain example embodiments—including a patient-by-patient basis and/or intra-patient basis—in order to evoke an appropriate therapeutic response from the patient. Such a response may be in the form of an efferent response, and/or an afferent response.

This Summary is provided to introduce a selection of concepts that are further described below in the Detailed Description. This Summary is intended neither to identify key features or essential features of the claimed subject matter, nor to be used to limit the scope of the claimed subject matter; rather, this Summary is intended to provide an overview of the subject matter described in this document. Accordingly, it will be appreciated that the above-described features are merely examples, and that other features, aspects, and advantages of the subject matter described herein will become apparent from the following Detailed Description, Figures, and Claims.

### SUMMARY OF FIGURES

FIG. 1 is a cross-sectional view of an upper portion of an airway passage in a patient.

FIGS. 2A and 2B illustrate reflex control of an airway in the patient.

FIG. 3 shows the connection between airway stability and negative pressure reflex (NPR).

FIG. 4 illustrates restoration of pharyngeal muscle tone by phrenic nerve stimulation that evokes NPR.

FIG. 5 is a cross-sectional view of a patient with a phrenic nerve system implanted with an implantable pulse generator (IPG).

FIG. 6 are charts showing variations over time of airway flow, respiratory flow, oxygen level and electrical simulation current applied to phrenic nerve.

FIG. 7 are charts showing variations over time of air passage flow rate and respiratory effort during a stimulated breath.

FIG. 8 is a flow chart for adjusting a parameter(s) for simulation of the phrenic nerve to treat sleep apnea for one embodiment.

FIG. 9 is an illustration of the linear and rotational accelerations that can be measured from a patient.

FIG. 10 showing a diagram where the linear and rotational accelerations are fused to generate a combined acceleration signal.

FIG. 11A shows the implantable stimulator with a built-in accelerometer.

FIG. 11B shows a high-level block diagram of an implantable stimulator, aka implantable pulse generator (IPG).

FIG. 12A is an illustration of transthoracic impedance measurement that is accomplished using a pair of electrodes under a configuration known as bipolar measurement. Furthermore, it illustrates the configuration where the excitation is provided from a time varying current source and the measurement is made in the form of a voltage waveform.

FIG. 12B is the electrical equivalent circuit of the configuration that is shown in FIG. 12A.

FIG. 13A is an illustration of transthoracic impedance measurement that is accomplished using a pair of electrodes under a configuration known as bipolar measurement, similar to that of FIG. 12A, except for the fact that the excitation is provided from a time varying voltage source, and the measurement is made in the form of an electrical voltage waveform.

FIG. 13B is the electrical equivalent circuit of the configuration that is shown in FIG. 13A.

FIG. 14A is an illustration of transthoracic impedance measurement that is accomplished using a set of three electrodes under a configuration known as tripolar measurement where the excitation is provided from a time varying current source and the measurement is made in the form of a voltage waveform.

FIG. 14B is the electrical equivalent circuit of the configuration that is shown in FIG. 14A.

FIG. 15A is an illustration of transthoracic impedance measurement that is accomplished using a set of four electrodes under a configuration known as quadripolar measurement where the excitation is provided from a time varying current source and the measurement is made in the form of a voltage waveform.

FIG. 15B is the electrical equivalent circuit of the configuration that is shown in FIG. 15A.

FIG. 16 is an illustration of an implant where a single lead is used for the delivery of the stimulation to the nerves governing the respiratory function and for the measurement of the transthoracic impedance.

FIG. 17 includes a schematic of an example transthoracic impedance measurement circuit and shows the excitation and measurement waveforms.

FIG. 18A is a graphical illustration of the train of bipolar excitation pulses used for the measurement of the transthoracic impedance.

FIG. 18B is a graphical illustration of the measured voltage of the transthoracic impedance measurement circuitry.

FIG. 18C is a graphical illustration of the imputed transthoracic impedance from the trace shown in FIG. 18B.

FIG. 19 is a high-level block diagram of the overall system that is used for the estimation of the air flow, ϕ(t), from the transthoracic impedance signal.

FIG. 20A is an illustration of the implantable system with a nerve stimulator and transthoracic impedance type sensor.

FIG. 20B is the simplified electrical block diagram of the implantable system shown in FIG. 20A where the system utilizes a transthoracic impedance sensor.

FIG. 21 shows a simplified electrical block diagram of an implantable system with a nerve stimulator and a set of dual sensors, namely a transthoracic impedance sensor and a set of accelerometers.

FIG. 22A shows the configuration of an implantable device with nerve stimulator, built in accelerometer and a lead separate than the stimulation lead for the measurement of the transthoracic impedance.

FIG. 22B shows the configuration of an implantable device with nerve stimulator, built in accelerometer and a lead that is used for nerve stimulation as well as for the measurement of the transthoracic impedance.

FIG. 23 shows the overall therapy system including the implantable stimulator, stimulation lead, transthoracic impedance sensing lead, a programmer and the cloud connection.

FIG. 24 shows a high-level block diagram of the implantable stimulator.

FIG. 25 shows the time domain signals recorded from a patient with sleep apnea without any stimulation.

FIG. 26A shows the time domain signals recorded from a patient with sleep apnea while the phrenic nerve is stimulated electrically.

FIG. 26B shows another stretch of time domain signals recorded from a patient with sleep apnea while the phrenic nerve is stimulated electrically.

FIG. 27 shows the 2×2 decision matrix used for the determination of air flow and effort to breathe.

FIG. 28A shows the mono-phasic waveform used for the stimulation of a nerve.

FIG. 28B shows the bi-phasic waveform used for the stimulation of a nerve.

FIG. 28C shows an illustrative example of a bi-phasic stimulation burst according to certain example embodiments.

FIG. 29 shows the timing of the stimulation waveform in relation to the phase of the respiratory cycle.

FIG. 30 shows the physiological effect of timing of the stimulation waveform in relation to the phase of the respiratory cycle.

FIG. 31 shows the flow-chart of the algorithm that can be used for the determination of the optimal timing of the stimulation waveform in relation to the phase of the respiratory cycle.

FIG. 32 shows the flow-chart of the algorithm that can be used for the determination of the optimal timing of the stimulation waveform in relation to the phase of the respiratory cycle.

FIG. 33 shows a case illustrating the start of entrainment as the stimulation rate is increased.

FIG. 34 shows a case illustrating the loss of entrainment.

FIG. 35 shows a flowchart of the algorithm used before entrainment.

FIG. 36 shows a flowchart of the algorithm used to initiate the entrainment.

FIG. 37 shows a flowchart of the algorithm used to maintain the entrainment.

FIG. 38 shows a therapeutic system delivering stimulation to multiple nerve targets.

FIG. 39 shows a high-level block diagram of the therapeutic system delivering stimulation to multiple nerve targets.

FIG. 40 shows an exemplary operation of a therapeutic system delivering stimulation to multiple nerve targets.

FIG. 41 shows the bidirectional stimulation of a nerve to activate the afferent and efferent fibers.

FIG. 42 shows the bidirectional stimulation of a nerve to activate the afferent and efferent fibers.

### DETAILED DESCRIPTION

In the following description, for purposes of explanation and non-limitation, specific details are set forth, such as particular nodes, functional entities, techniques, protocols, etc. in order to provide an understanding of the described technology. It will be apparent to one skilled in the art that other embodiments may be practiced apart from the specific details described below. In other instances, detailed descriptions of well-known methods, devices, techniques, etc. are omitted so as not to obscure the description with unnecessary detail.

Sections are used in this Detailed Description solely in order to orient the reader as to the general subject matter of each section; as will be seen below, the description of many features spans multiple sections, and headings should not be read as affecting the meaning of the description included in any section.

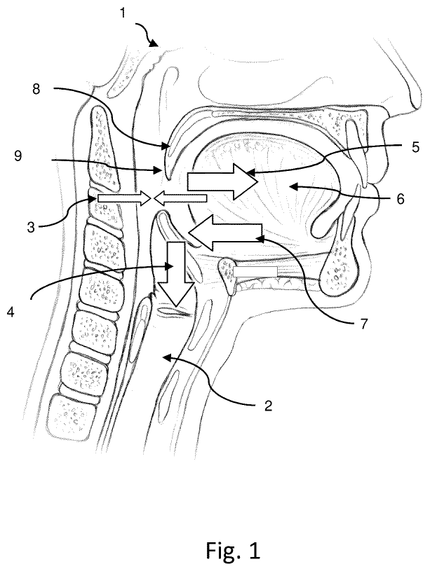

### Description of FIGS. 1-4: Introduction

Example techniques discussed herein can augment the afferent limb of a pharyngeal mechanoreflex, for example a Negative Pressure Reflex (NPR) may be triggered, that naturally dilates and stabilizes the airway in response to increased negative transmural pressure in the airway. Decreases in NPR during sleep may contribute to snoring and airway collapse in at least some OSA patients.

In healthy people during wakefulness, pharyngeal patency is protected by dilator muscles, with negative airway pressure (collapsing pressure) acting as a local stimulus for their graded activation. The respiratory pump of a person can be modelled as a bellow or a pneumatic cylinder where the rapid descent of the diaphragm creates an inrush of fresh air through the nose and down the airway into the lung. This airflow creates a pressure gradient (e.g., that is significant) along the airway that escalates with the increase of the upstream resistance. Since the airway is a collapsible tube, force exerted by this negative pressure during inspiration needs to be opposed to prevent collapse. This opposition is the primary role of the NPR.

The NPR can manifest naturally by robust and very rapid (within 30-50 milliseconds) activation of pharyngeal dilator muscles when a rapid pulse of suction (negative) pressure is applied by inspiration of ambient air through the nose. Such activation can be a protective reflex that allows the pharynx to resist closure during a potentially collapsing perturbation such as eating, vocalizing, sniffing, or gasping for air.

In connection with certain example embodiments, afferent feedback through the NPR can lead to a coordinated response in multiple accessory muscles that maintain pharyngeal patency without arousing the patient from sleep. Example embodiments described herein include techniques involving reflexes that can be used for therapy and implemented in, for example, embedded software algorithms using illustrative hardware and implantation procedure(s).

In connection with some examples, the approach to triggering NPR to treat OSA disclosed herein can be counterintuitive and goes against some entrenched beliefs and clinical practices. First, negative airway pressure causes the airway to collapse and the approach of stimulating the phrenic nerve will increase negative pressure in the airway. It is counterintuitive to increase negative pressure to open an airway. Second, clinical practice of phrenic nerve stimulation in individuals with central neurologic disease such as congenital hypoventilation required tracheostomy to prevent airway collapse induced by augmented negative pressure. Third, when a healthy individual is placed in a negative pressure ventilator, e.g., an iron lung, their normal respiratory effort and central chemoreflex cause a reduction or elimination of ventilatory drive. While NPR is mostly preserved and protects their airway from collapse, it was observed that in individuals with OSA, the use of negative pressure ventilation increased collapsibility of the airway. It is likely that these considerations prevented use of NPR to stabilize the airway during sleep in research or clinical practice.

Breaking with tradition and prevailing concepts, example techniques propose to create or enhance negative pressure conditions in an airway to trigger NPR to treat airway collapse. In some embodiments, techniques are applied to a patient that restores the NPR in a patient with OSA during sleep by periodically stimulating one or both phrenic nerves. In some examples, this results in generating contractions of the diaphragm. In some examples, the contractions may be vigorous and/or relatively short (for example, less than 50% of duration of the natural breath) and/or generally coincide with the inspiratory part of the respiratory cycle and more specifically with a late expiration—early inspiration period.

In some examples, nerve firing augmentation may increase afferent signal above the threshold that forces the respiratory central control center to generate efferent signals to various groups of dilator muscles sufficient to stiffen the airway and restore airflow. In this context, if stimulation bursts occur frequently, for example at a natural respiratory rate of 6 to 20 per minute, the airway does not stay closed long enough to impede (e.g., significantly) ventilation or gas exchange and oxygen saturation is maintained. Thus, it may be possible and/or desirable to synchronize the diaphragmatic contraction to the patient-initiated inspiration or to set the rate and allow patient to synchronize to the stimulation. In some embodiments only every second or other ratio of breaths are stimulated.

In some examples, phrenic nerve stimulation (PNS) can be used to bias or offset the diaphragm. Or, more generally, to break expiration, thereby producing moderate dynamic lung hyperinflation. This modality of stimulation may be especially efficacious in patients with reduced lung volume. In patients with reduced lung volume, restoring lung volume may contribute to airway patency.

In some cases, sleep-induced decrements in lung volume can lead to reductions in longitudinal traction on the airway, yielding an increasingly collapsible pharynx even in the patients with normal lung volume while awake. Some individuals may be quite dependent on this mechanism to maintain airway patency while awake and lose it during sleep. In some examples, lung volume biasing may be combined with periodic contractions evoking NPR in some patients.

In some instances, lung volume can be increased “statically” by biasing of the lung by the application of constant low-level tone to the phrenic nerve, which prevents complete lung deflation, and exerts caudal traction and stiffens the pharynx.

In some instances, lung volume can also by trapped by “expiratory breaking” by increased frequency of phrenic nerve busts or increased inspiratory to expiratory (I:E) ratio, which traps air “dynamically” and prevents complete lung deflation to exert caudal traction and stiffen the pharynx.

Obstructive sleep apnea (OSA) is the intermittent cessation of breathing during sleep due to the collapse of the pharyngeal airway. A purpose of OSA therapy can be to increase tension of muscles that support the pharynx and prevent it from collapsing.

Pharynx (also called in this patent pharyngeal airway or for simplicity just the “airway”) is a tube that connects nasal and oral cavities to the larynx and the esophagus. It is separated into nasopharynx, oropharynx, and laryngopharynx. The pharynx is a muscle tube that is collapsible at any point along the way. There are 20 or more muscles surrounding the airway and actively constricting and expanding the upper respiratory tract lumen. These muscle groups also contribute to the stiffness of the airway, defined as its ability to withstand negative transmural pressure regardless of its caliber. As used herein, “airway stabilization” means the stiffening of the airway by mechanical or neural intervention.

Airway muscles can be divided into four groups: muscles that regulate the soft palate position (ala nasi, tensor palatini, levator palatini); tongue (genioglossus, geniohyoid, hyoglossus, styloglossus); hyoid device (hyoglossus, genioglossus, digastric, geniohyoid, sternohyoid); and posterolateral pharyngeal walls (palatoglossus) pharyngeal constrictors). These muscle groups can interact to keep the airway open and closed. Soft tissue structures form the walls of the upper airway and tonsils include: soft palate, uvula, tongue, and lateral pharyngeal walls

In some cases, the site of the airway collapse is significant in the pathophysiology of OSA and in targeting any therapy to prevent collapse. Airway collapse sites that are commonly identified in literature are associated with:

Retrolingual space (tongue base), Velopharyngeal space (Soft palate occlusion) and/or Hypopharyngeal space (lateral airway wall occlusion)

Turning now to FIG. 1, a cross-sectional view of an upper portion of an airway passage in a patient is shown. This figure illustrates the balance of forces that keep an airway open during inspiration. Inspiratory negative pressure and extraluminal positive pressure tend to promote pharyngeal collapse. Upper airway dilator muscles and increased lung volume (as it fills with air) tend to maintain pharyngeal patency. Patient 1 inhales air at the atmospheric pressure through the nostrils. Inhaled air travels down the pharyngeal airway 2. Soft pallet 8 (sometimes called vellum) defines the velopharynx or velopharyngeal space 9 that is the most common location of the airway collapse.

Variables tending to promote pharyngeal collapse include negative pressure 3 within the airway and positive pressure 4 outside the airway. It is the product of pressure caused by posture and gravity, fat deposition, and other anatomic factors such as small mandible 6. The sum of these pressures defines the transmural pressure sensed by mechanoreceptors in the airway. Negative inspiratory pressure 3 is dynamic and present during inspiration at any point along the airway. It is proportional to airflow and upstream resistance. Conversely, patency is preserved by activation of pharyngeal dilator muscles 5 (e.g. genioglossus) and by increases in lung volume 7, which tend to keep the airway open by longitudinal traction. As a result, dilating forces (muscle activation) have a complex interaction with collapsing forces generated by anatomy and airway negative pressure.

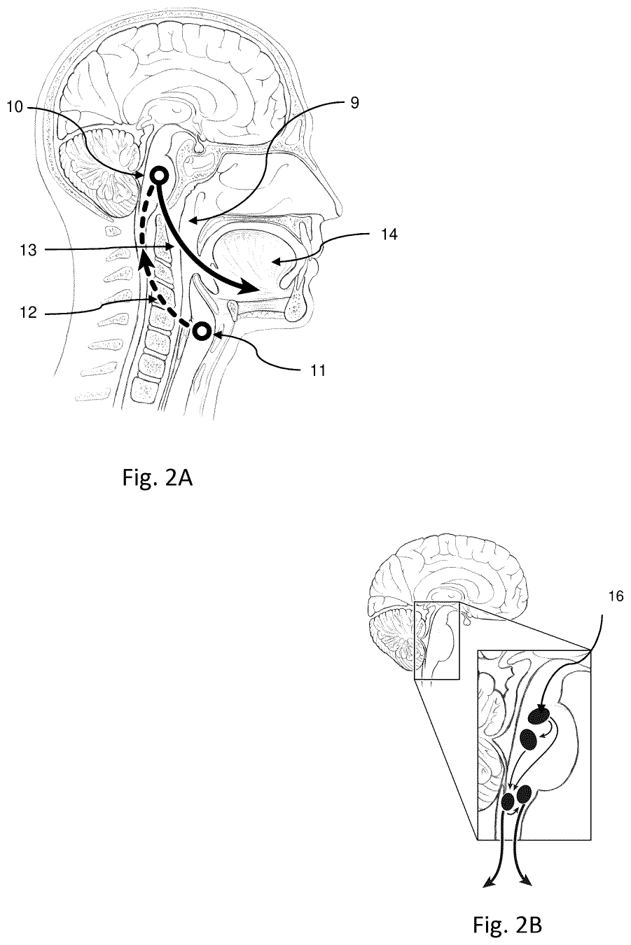

FIGS. 2A and 2B illustrate reflex control of the airway. The central neural system (CNS) pattern generator (respiratory center) 10 is located in the medulla 16 of the brain. The rhythmicity center of the medulla in the brain-stem controls automatic breathing during sleep and consists of interacting neurons that fire either during inspiration (I neurons) or expiration (E neurons). I neurons stimulate neurons that innervate respiratory muscles (to bring about inspiration). E neurons inhibit I neurons (to ‘shut down’ the I neurons & bring about expiration). Apneustic center (located in the pons) stimulate I neurons (to promote inspiration). Pneumotaxic center (also located in the pons) inhibits apneustic center & inhibits inspiration. This inhibition can be overrun by phrenic nerve stimulation that affects the respiratory pump directly.

The respiratory center 10 receives inputs from physiologic sensors 11 via various afferent sensory nerve fibers and maintains a patent airway through stiffening and dilation by synchronized contraction and relaxation of muscles via efferent motor fibers. The airway dilator muscles include the genioglossus 14 that protrudes and retracts the tongue. The genioglossus has a direct effect on the velopharyngeal space 9 where airway occlusion often occurs. Such physiologic feedback arrangement is known as a closed loop reflex. Generally, such reflexes are known as “autonomic” since they do not depend on consciousness.

Negative Pressure Reflex (NPR) may be one example of a pharyngeal mechanoreflex activating dilator muscles. A mechanoreflex is a reflex triggered by stimulation of a mechanoreceptor. A muscle spindle stretch receptor, pressure receptor, a sheer stress receptor or flow receptor can be an example of a mechanoreceptor that reacts to mechanical perturbation, such as deformation and generates afferent neural signal consisting of a train of action potentials in a bundle of nerve fibers.

NPR is a physiologic reflex that can be used in connection with certain examples. NPR can manifest naturally during every breath by robust and very rapid (within 30-50 milliseconds) activation of pharyngeal dilator muscles when a rapid pulse of suction (negative) pressure is applied through the nose and sensed by transmural pressure sensors in the pharyngeal mucosa. In connection with some examples, NPR can be enhanced or induced by electric stimulation of phrenic nerves that causes diaphragmic contraction. The magnitude of the signal sensed by sensors 11 can be based one or proportionate to the intensity of diaphragmic contraction and the upstream resistance of the airway, particularly in the velopharyngeal space 9.

If the airway is occluded, then pressure will generally become more negative and the afferent limb traffic of the reflex becomes much stronger. The response of the CNS center 10 is in turn proportionate to the input from the afferent limb 12. This response generates stronger output in the efferent limb 13 which results in the stronger contraction of the dilator muscles 14. Ultimately the entire closed loop response becomes strong enough to open the airway and allow air in. This in turn leads to the reduction of negative pressure and the sensed signal in the afferent limb 12. The closed loop system comes to the steady state and respiratory stability can be restored.

FIGS. 1, 2A, and 2B illustrate elements of pharyngeal anatomy and innervation. Because of the physiological importance of maintaining pharyngeal patency and the many tasks required of this portion of the airway (speech, swallowing, etc.), a sophisticated motor control system has evolved, with more than 19 upper airway muscles playing a part. The following paragraphs expand the complexity of this natural arrangement for maintaining the airway open and prior attempts to improve it in OSA patients.

During natural inspiration, negative intra-luminal pressure pulls three soft tissue elements, the tongue, posterior pharyngeal walls, and soft palate, toward each other, thereby reducing the airway lumen in the velopharyngeal region. This airway-collapsing action is opposed by pharyngeal dilator muscles, including the genioglossus, geniohyoid, and tensor and levator veli palatini. Additionally, activation of the pharyngeal constrictors stiffens the airway walls.

The soft palate (the velum) comprises muscle and tissue, which makes it mobile and flexible. When a person swallows, the soft palate rises to seal the opening of the airways to prevent pressure from escaping through the nose. The shape, position, and movements of the soft palate are maintained by five pairs of muscles, including tensor veli palatini (TVP), levator veli palatini (LVP), palatopharyngeus (PP), palatoglossus (PG), and musculus uvula (MU). The tensor veli palatini muscle (tensor palati or tensor muscle of the velum palatinum) is a broad, thin, ribbon-like muscle in the head that tenses the soft palate.

The tensor veli palatini is supplied by the medial pterygoid nerve, a branch of mandibular nerve, the third branch of the trigeminal nerve—the only muscle of the palate not innervated by the pharyngeal plexus, which is formed by the vagal and glossopharyngeal nerves. The tensor veli palatini tenses the soft palate and by doing so, assists the levator veli palatini in elevating the palate to occlude and prevent entry of food into the nasopharynx during swallowing.

The palatoglossus muscle functions as an antagonist to the levator veli palatini muscle. Palatoglossus arises from the palatine aponeurosis of the soft palate, where it is continuous with the muscle of the opposite side, and passing downward, forward, and lateral in front of the palatine tonsil, is inserted into the side of the tongue, some of its fibers spreading over the dorsum, and others passing deeply into the substance of the organ to intermingle with the transverse muscle of tongue. It is innervated via vagus nerve (via pharyngeal branch to pharyngeal plexus). It elevates posterior tongue, closes the oropharyngeal isthmus, and aids initiation of swallowing. This muscle also prevents the spill of saliva from vestibule into the oropharynx by maintaining the palatoglossal arch.

The genioglossus muscle (GGM) receives input from the brainstem respiratory central pattern generator via the Hypoglossal Nerve (HGN). The presence of ‘pre-activation’ (hypoglossal nerve firing 50-100 ms prior to the phrenic nerve) supports the presence of pre-motor inputs to the hypoglossal motor nucleus in the medulla.

While successful in some, HGN stimulation is not an effective solution for some patients. In some cases, effectiveness could be restored by increasing the power applied to the nerve, but many patients cannot tolerate the increased power regiment for one reason or another. A possible reason for this is that the acceptable level of GGM activity is not sufficient to overcome other physiological changes that occur and persist during sleep, such as low activity of the other dilator muscles, altered co-activation patterns with the other dilator muscles and low lung volumes that results in the reduced caudal traction of the airway. These limitations are addressed in this application through novel approaches such as manipulation of lung volume and transmural airway pressure via stimulation of phrenic nerve.

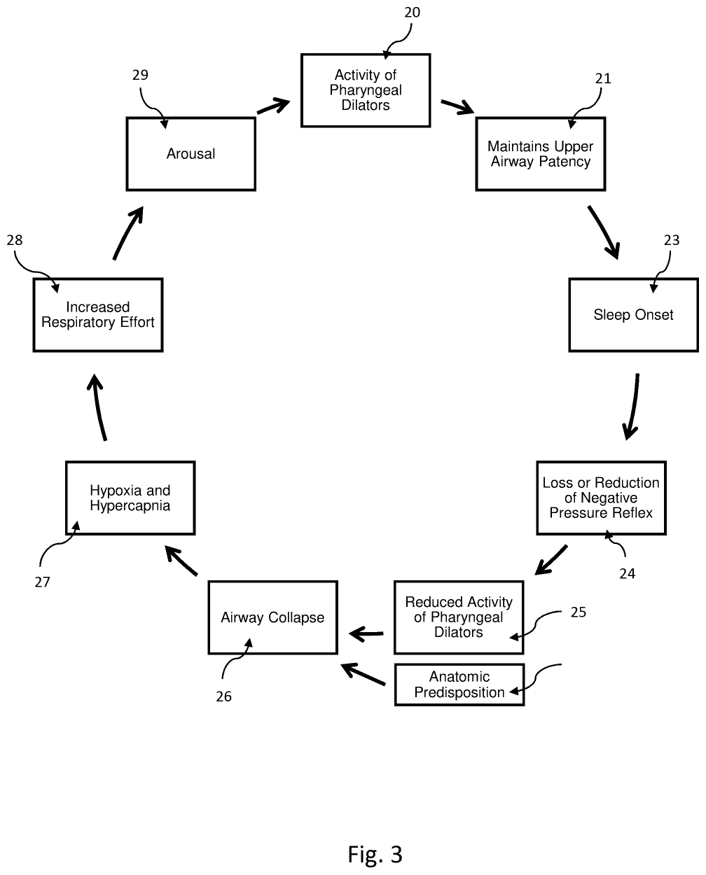

FIG. 3 further illustrates the role of NPR in the pathogenies of OSA. In healthy persons and unhealthy OSA patients during wakefulness, pharyngeal patency 21 is maintained by the phasic activation of pharyngeal dilator muscles 20, with negative airway pressure (collapsing pressure) acting as a local stimulus to their activation. The negative pressure reflex is a protective reflex that allows the pharynx to resist closure during a collapsing perturbation. The dilator muscles respond within tens of milliseconds to negative pharyngeal pressure, thereby maintaining airway patency.

To overcome compromised pharyngeal anatomy 22, such as in common obesity, suboptimal tongue, or mandible anatomy etc., the upper airway dilator muscles of a patient with OSA must be more active during wakefulness than those of healthy individuals. In wakefulness NPR responds to the increased (more negative) negative pressure in patients with compromised anatomy. The sensed response is a product of the smaller pharyngeal lumen and the need for greater intrapharyngeal pressure to generate adequate airflow. This increased negative pressure drives greater muscle activation. Thus, the airway muscles compensate for the deficient anatomy of the OSA patient while awake, and their ventilation is maintained. Even in patients with very severe OSA, disordered breathing events occur only during sleep, emphasizing the importance of central control in the pathogenesis of this disorder.

Neuromuscular reflexes can be reduced 24 during sleep 23. The ability of the pharyngeal dilator muscles to respond to negative pressure is substantially attenuated during sleep even in healthy people. Loss of these excitatory inputs to the efferent hypoglossal motoneurons may greatly decrease the ability of the genioglossus and other upper airway dilator muscles to respond to negative pressure 25 compared to wakefulness. Loss or reduction of this reflex mechanism during sleep would be expected to precipitate large decrements in muscle activity and subsequent airway closure 26. As a result, if an individual's pharyngeal anatomy is compromised, their airway will be unprotected by NPR and vulnerable to collapse during sleep. In OSA, airway closure can lead to hypoxia and hypercapnia 27, which evoke CNS chemoreflex. Unlike mechano-reflexes such as NPR, chemoreflexes depend on the blood circulation for response and may take as long as 15 to 90 seconds to produce the response from the respiratory pump 28 and increased respiratory effort. These delays manifest as periodic breathing and apnea hyperpnea cycles. Ultimately increased respiratory effort is often accompanied by arousal 29 and restoration of wake level of activity of pharyngeal dilators 20. As cycle repeats itself as frequently as 20 to 90 times an hour patient's sleep can become compromised.

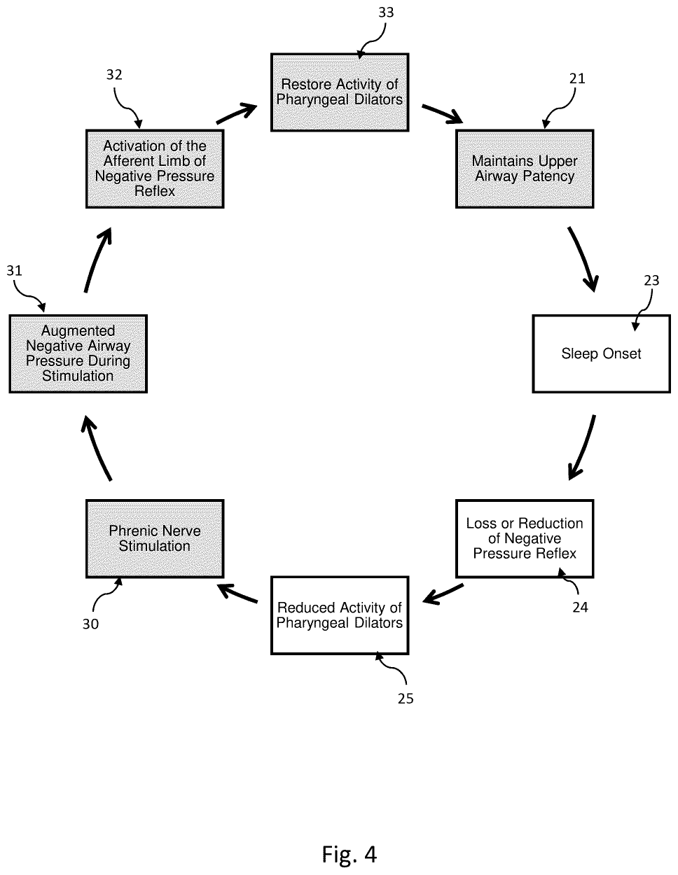

### Description Of FIGS. 4-8: Stimulation Examples

The respiratory system of an individual may have different (e.g. many) parallel control mechanisms for controlling various aspects of bodily function. An example of such a control mechanism include the body's negative pressure reflex (NPR) that is discussed herein. Alternative, or additional, control mechanisms that may be present in the body may include a direct afferent pathway (which also may be called a “physiologic pathway”) that may operate with signals being communicated directly to the brainstem from triggering a given nerve or nerves (e.g., the phrenic nerve and/or other nerves). This direct afferent pathway may thus allow a triggered nerve(s) to directly “message” the brainstem, which may then activate one or more functions of the body.

Any or all of the techniques discussed herein may be applicable in the context of one or more of the body's control mechanisms. An illustrative example, the stimulation of the phrenic nerve may cause NPR in a sleeping individual in some examples. In other examples (either with the same or different stimulation), a stimulated nerve may result in a signal being communicated (e.g. to the brainstem), which may then trigger movement of one or more muscles in a sleeping individual. In certain examples, the resulting stimulation of afferent nerves may be different from efferent nerves. For example, in the case of NPR, contraction of the diaphragm may be triggered. In the case of triggering a direct afferent pathway signal, the diaphragm of the patient may not be recruited. Instead, other muscles may be triggered/recruited by the brain in order to cause an opening (e.g., which may include further opening) of the airway of the patient. Note that in certain cases, individuals may be more or less tolerable to the triggering of one or more of the control mechanisms being triggered. Accordingly, selecting/triggering a given control mechanism may be provided according to the techniques discussed herein.

Turning now to FIG. 4 the restoration of pharyngeal muscle tone by phrenic nerve stimulation is shown. In this example, the negative pressure reflex of a patient is shown. Sleep onset 23 inevitably leads to reduction of natural NPR 24. The reduced reflex leads to the reduction of naturally occurring periodic efferent limb signaling to muscles responsible for maintaining airway patency 25. This results in greatly increased inspiratory airway resistance and likely intermittent airway collapse.

Therapy includes the periodic stimulation of the phrenic nerve 30. This can result in a diaphragmic contraction. In some examples, the diaphragmic contraction can be a robust and/or vigorous response. In some examples, the contraction may be smaller and/or more targeted in nature (as discussed in greater detail herein). For example, the contraction may be timed (e.g., in relation to the patient's respiratory cycle, for example, just before the beginning of inspiration, expiration, or other point) to generate a sufficient response in the patient. The diaphragmic contraction can immediately (within tens of milliseconds) generates negative pressure 31 within the airway. This pressure change is picked up by transmural pressure sensors in the pharyngeal mucosa and the afferent limb of the NPR is potentiated 32. This leads to the reflex activation of the efferent limb and contraction of dilators 33 which restores airway patency. Since NPR is very fast, this process can be cyclically repeated at a rate consistent with natural breathing (6 to 20/min). As a result of this periodic activity airway never stays closed long enough to evoke hypoxia and activate respiratory chemoreflex. It generally takes an OSA patient tens of seconds of apnea to desaturate by 3-4% oxygen saturation which is considered clinically significant. The oscillatory cycle of apnea-hyperpnea does not occur or is greatly attenuated and sleep disruption is prevented.

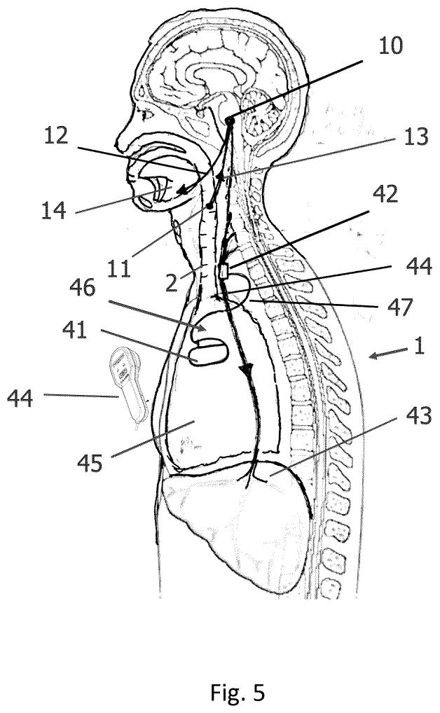

FIG. 5 schematically illustrates one physical embodiment of the invention. Patient 1 is implanted with a nerve stimulation system 46 including an implantable pulse generator (IPG) 41 that is electrically connected to the electrode system 42 that is implanted in proximity of the phrenic nerve 44. Stimulation burst from the IPG generates a response in the patient that may be a contraction and/or descent of the diaphragm 43. The contraction may be vigorous in nature in certain instances or may be smaller in some instances. Indeed, for the same patient different stimulations may vary the nature of the contraction that is being triggered. In any event, the contraction of the diaphragm fills the lung 45 with air and generates negative pressure in the airway 2. Negative pressure is sensed by the receptor 11 that activates the afferent limb of the MPR. Respiratory center 10 responds by generating efferent signal 12 that activated the dilator muscles illustrated by genioglossus 14. It is understood that other dilator muscles are also co-activated. The airway is dilated and stiffened by synchronized effort of muscle groups activated by the reflex. Negative pressure is strongest if the airway is occluded and that facilitates removal of the obstruction.

Stimulation of the phrenic nerve can assist or replace natural breathing. As shown in FIG. 5, a phrenic nerve stimulation system 46 can include an electrode sub-system 42 adapted to apply electric current to one or two phrenic nerves 44 in a pattern that is programmed or other stored in memory and used by a microprocessor inside the Implanted Pulse Generator (IPG) 41. The illustrative system can include a lead 47 electrically connecting the IPG and the electrode. In some examples, the system can be configured to determine or sense respiration states (inspiration, expiration), sensing of airflow, chest motion, or pressure.

In some examples, the electrode system 42 may be a nerve cuff, an endovascular electrode, a paddle electrode, or a percutaneously inserted wire electrode approximating phrenic nerve in the neck or in the chest. It may be connected to the IPG or a subcutaneous wireless antenna in communication with an EPG (External Pulse Generator—not shown) by a flexible lead 47. Stimulation can be monopolar, bipolar, or multipolar and apply energy to either or both right and left phrenic nerves. The IPG can include an implanted battery, rechargeable or single use, or receive energy wirelessly by a transdermal RF link from an external device outside of the body. It can be equipped with telemetry such as Bluetooth.

In some examples, an IPG/EPG can include a microprocessor with non-transitory memory and other associated circuitry that is configured to execute embedded software/firmware that is used to activate/deactivate the device. In some examples, a user interface may be provided to allow for adjustment of stimulation parameters (also called stimulation characteristics) such as current, voltage, pulse duration and frequency, pulse burst rate, duty cycle, and/or burst shape. Control may be carried out via wireless communication using a programmer or “wand” 44 that can modify the embedded software and upload and download data to the IPG when brought within close distance with the patient's body.

Phrenic nerve stimulation (PNS) can improve airway patency through the physiologic mechanism of activation of a mechanoreflex such as the NPR and by the increase of lung volume, which are compatible and can be embedded in one hardware system. Certain example implementation may involve a compromise between effectiveness of the therapy and the ability of the patient to tolerate therapy. For example, and in some cases, the effectiveness of therapy may be proportional to electric field energy applied to the nerve by the pulse generator (PG). The PG generates electric current pulses that generate action potentials in the targeted nerve fibers that innervate targeted muscle fibers. Often untargeted nerve fibers are also activated limiting patient's tolerability. The tolerability may include many factors such as pain, muscle twitching, unpleasant sensations and interference with respiratory mechanics, gas exchange and sleep quality. The embedded software in the IPG can include features needed to titrate energy to achieve compromise between effectiveness and tolerability.

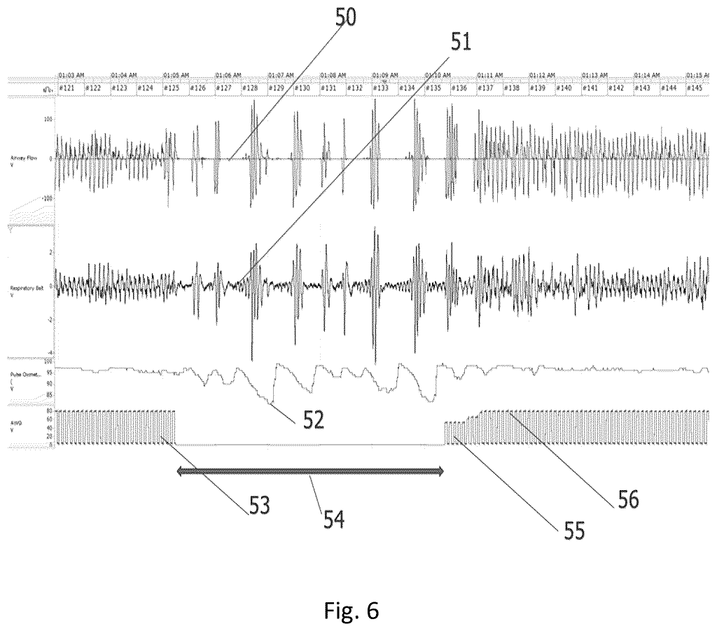

FIG. 6 includes charts showing therapy results and artificial nerve stimulation over a period in which an example therapy is being applied to a patient with OSA.

During the control time period 54, stimulation is turned off. During this period the patient experienced severe OSA, as evidenced by the absence of airflow (first trace from the top) during apnea periods 50, presence of respiratory effort 51 (second trace from the top) during apnea and oxygen desaturations 52 (third trace from the top). Oxygen desaturation periods 52 follow apnea periods 50 after a circulatory delay. Oxygen desaturation and accompanying rise of CO₂ enables chemoreflex to arouse the patient and terminate apnea period by restoring airway patency. This is the cycle naturally occurring during OSA and illustrated by FIG. 3. When stimulation is turned on during periods 53, 55 and 56 patient's breathing is vastly improved. Stimulation bursts of sufficient magnitude evoke reflexes that likely include the NPR and open the airway almost instantly without awakening the patient as illustrated by FIG. 4. Blood gases O₂ and CO₂ are maintained, and patient does not experience significant periodic breathing or long periods of apnea.

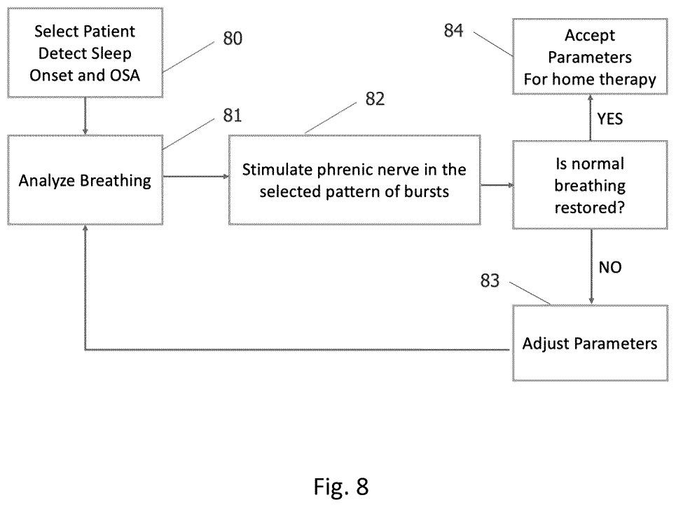

Increasing the energy level of phrenic nerve stimulation (e.g., the amplitude of the stimulation energy) from level 55 to level 56 resulted in a gradually more complete resolution of airway obstruction. It will be appreciated that it is desirable to have a stimulation level that is high enough to open the airway of the patient, but not too high to cause discomfort or arousal of the patient. This process is further illustrated by FIG. 8.

For the therapy shown in FIG. 6, the patient is wearing a nasal musk attached to a precision air flow meter and is equipped with thoracic and abdominal respiratory belts, finger pulse oximeter and standard polysomnography (PSG) montage as commonly used during sleep studies. Percutaneous electrode was inserted in their neck close to the left phrenic nerve and connected to a bedside electric pulse generator operating in constant current mode. Bipolar pulse trains of 150 microseconds long square pulses were applied at the current of 1 to 5 mA, at 30 Hz. Adjustments are made by the operator to the stimulation current in 0.25 mA increments to achieve the desired stabilization of breathing.

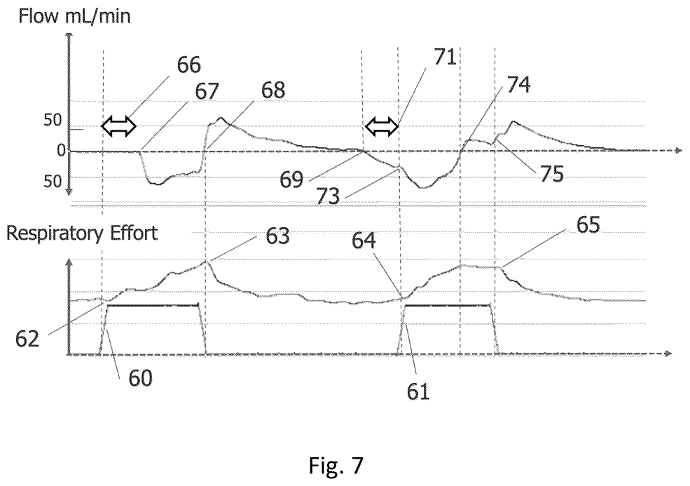

FIG. 7 are charts of airflow rate and respiratory effort over time for two breaths from the same patient during a portion of the therapy period illustrated in FIG. 6. Stimulation bursts 60, 61, e.g., called pulse trains, are applied at a rate (called a stimulation rate herein) that approximates a patient's natural breathing rate. The stimulation bursts in this case have a duration of approximately equal to ⅓ of the breath (duty cycle of 33%). The first stimulation burst 60 is initiated when the patient's airway is closed as evidenced by air flow of zero during period 66. An airflow of zero indicates that the airway is collapsed, the patient is asleep, and their base pharyngeal muscle tone is not sufficient to keep their airway open.

As shown in FIG. 7, the airway opens abruptly, and inspiratory airflow starts 67 after a time delay 66. This time delay is the time it took the respiratory effort of the diaphragm, natural and stimulated, and negative pharyngeal pressure to reach the afferent signal threshold that activated the reflex opening. Patient then inspires at the peak airflow rate of >50 ml/min, which indicates unobstructed airway. The low trace illustrates abdominal circumference indicative of inspiratory effort (diaphragmic excursion). Beginning of effort 62 coincides with the stimulation 60 but precedes inspiratory airflow 67 by the delay 66. Inspiration stops and turns into expiration at the point 63 when the central control initiates exhalation phase of breath and stimulation is turned off.

The next breath is initiated by the respiratory center of the patient. The airway is obstructed but not closed, as evidenced by airflow 69. The airflow is limited by airway resistance and peaks at approximately 30 ml/min. Inflection point 73 coincides with the onset of the second stimulation burst 61 after the delay time 71. Airflow is accelerated and abdominal excursion indicates significant diaphragmic contraction (effort). Inspiration is terminated by the respiratory center at the point 74 where airflow is reversed and becomes exhalation at a modest rate. This indicates that only one lung is exhaling. The lung controlled by the stimulated phrenic nerve only exhales at the inflection point 75 where expiratory flow accelerates. It coincides with the termination of the stimulation burst 61 and cessation of effort 65.

FIG. 8 illustrates a process of therapy selection that may be implemented in software/firmware that is used to control one or more aspects of therapy as discussed herein. Initially, a patient can be identified to have moderate or severe OSA based on standard home PSG test. For example, patient may have apnea hypopnea index (AHA) ≥20 events per hour. Patient is implanted with an IPG and a phrenic nerve stimulation electrode. IPG is confirmed operational, and patient is discharged for a period needed to heal, such as one month. Patient is brought to the office of the sleep physician specialist for therapy activation.

Once in the office, the process shown in FIG. 8 may be performed. While patient is sleeping in the office 80 their breathing pattern and sleep pattern are analyzed by standard instrumentation used for sleep studies 81. Stimulation of phrenic nerve is initiated at an initial set of parameters 82. Rate can be a number close to patients natural breathing or a different reasonable rate comfortable for the patient. Duty cycle (burst duration) can be set to I:E ratio of 1:3 and stimulation current is gradually increased until diaphragmic contractions corresponding to stimulation bursts are clearly detected.

If the patient's OSA is resolved and AHI is reduced by at least 50%, patient may be sent home 84 with a selected set of parameters and instructions to initiate therapy every night. If not, parameters can be changed 83 and titrated upwards until OSA is resolved. For example, stimulation current (index of energy delivered to the nerve) can be increased. Increased current generally results in stronger diaphragmic contractions until muscle fibers are fused and the muscle cannot contract more. The rate at which amplitude of pulses in the bust is increased, often called ramp time, can also be shortened to generate (e.g., a more vigorous or abrupt) diaphragmic contractions.

Duty cycle and/or stimulation rate can also be increased with the understanding that some air trapping may occur during stimulation if stimulation bursts are more frequent or last longer. Some patients may benefit from lung volume increase during sleep to prevent lung collapse and loss of caudal traction exerted by the lung inflation on the airway. All stimulation parameters ae titrated based on patient's tolerance. It is anticipated that after patient adapted to therapy, the intensity of stimulation may be increased.

In certain examples the timing of the stimulation can be synchronized to the phase of the respiratory cycle. In certain cases, such synchronization may be achieved in a clinical environment. However, in a home setting such synchronization may be more difficult to achieve when a patient has an implantable stimulator. To provide the implantable device with information regarding the phase of the respiration, various sensory systems, including implantable accelerometers and transthoracic impedance sensors, can be used in accordance with certain examples.

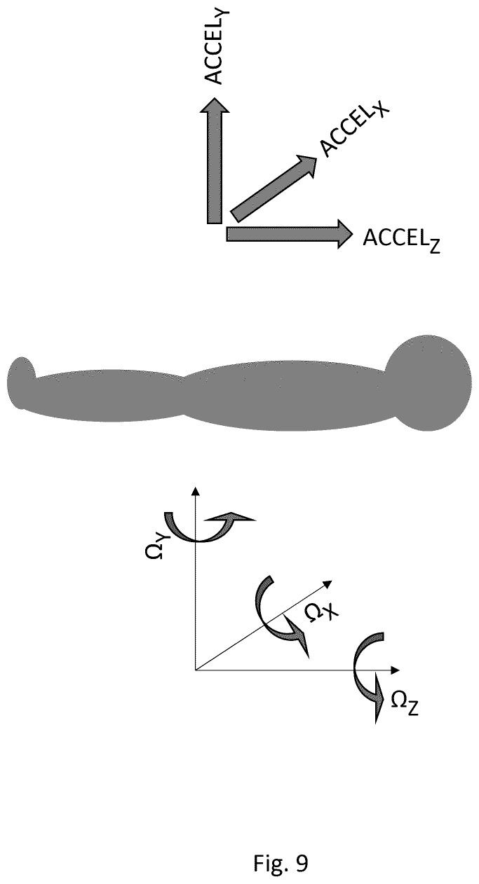

### Description Of FIGS. 9-11B: Accelerometer Sensor

FIG. 09 shows the six motions of the human body that can be measured by an implantable 6-axis accelerometer, which are 1) ACCEL_{X}: Linear acceleration in lateral direction; 2) ACCEL_{Y}: Linear acceleration in anterior—posterior direction; 3) ACCEL_{Z}: Linear acceleration in inferior—superior direction; 4) GYRO_{X} (Ω_{X}): Rotational acceleration around the lateral axis; 5) GYRO_{Y} (Q_{Y}): Rotational acceleration around the anterior—posterior axis; and 6) GYRO_{Z} (Q_{Z}): Rotational acceleration around the inferior—superior axis.

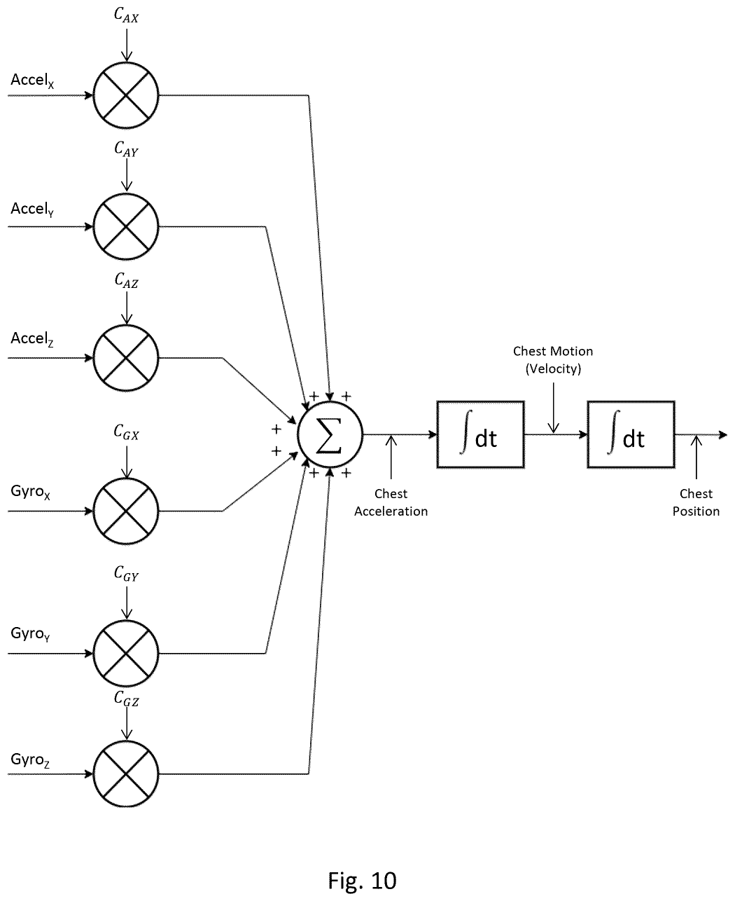

These acceleration signals can be fused (e.g., combined) together to generate a single chest acceleration signal, as shown in FIG. 10. Coefficients C_{AX}, C_{AY}, C_{AZ}, C_{GX}, C_{GY} and C_{GZ} can be individualized for each patient to fuse the linear and rotational acceleration signals to form a single chest acceleration signal. Integration of the chest acceleration signal can be used to determine or calculate chest motion and chest position, which in turn can be used for the estimation of the respiration.

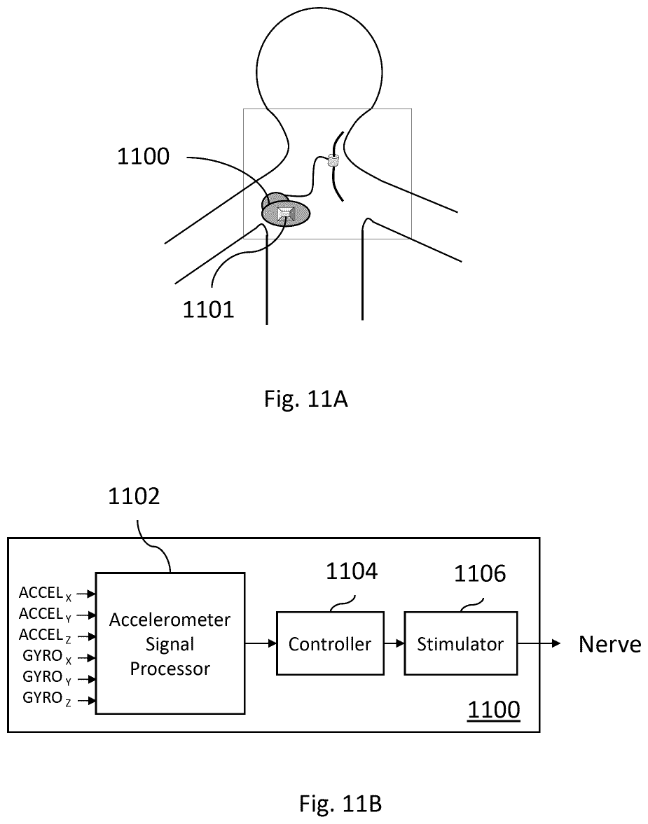

FIG. 11A shows an example implanted pulse generator 1100 with a phrenic nerve stimulation lead as well as its onboard accelerometer 1101.

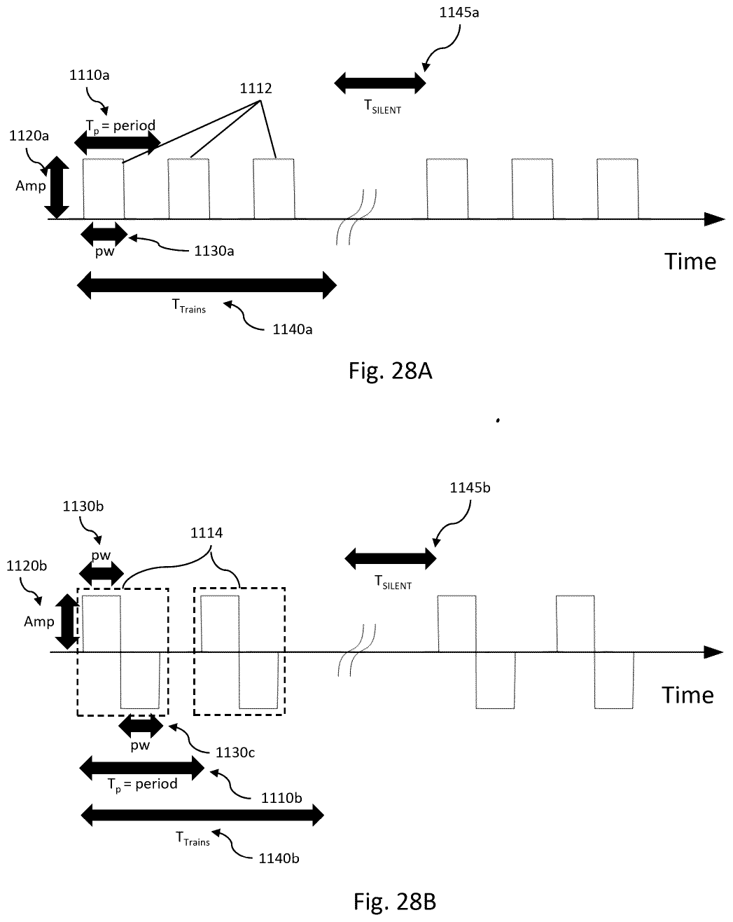

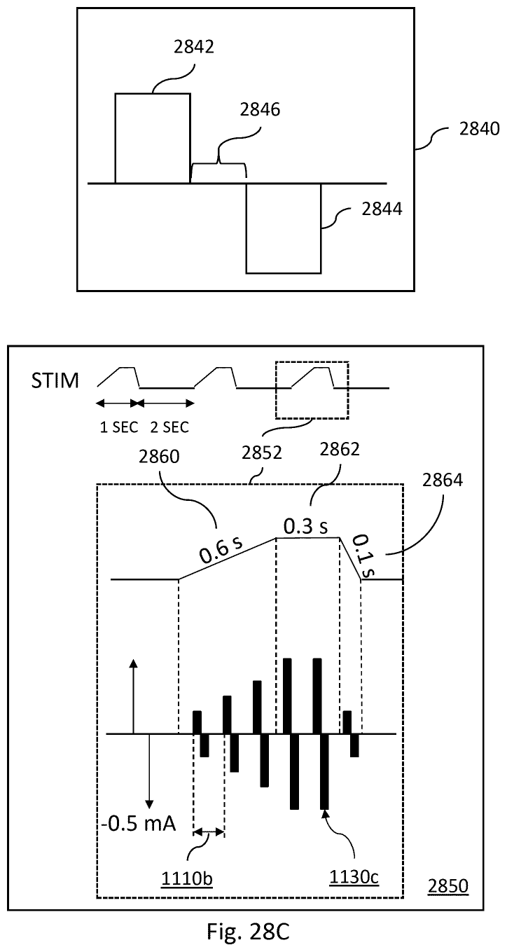

FIG. 11B shows a high-level block diagram of the implantable pulse generator 1100 shown in FIG. 11A. Raw acceleration signals are processed by the Accelerometer Signal Processor 1102 shown in FIG. 10. Output of the Accelerometer Signal Processor is used by the Controller 1104 to estimate the current phase of the respiratory cycle. Ultimately, the Controller 1104 triggers the Stimulator 1106 to generate the output pulses which will be described later and illustrated in FIGS. 28A and 28B.

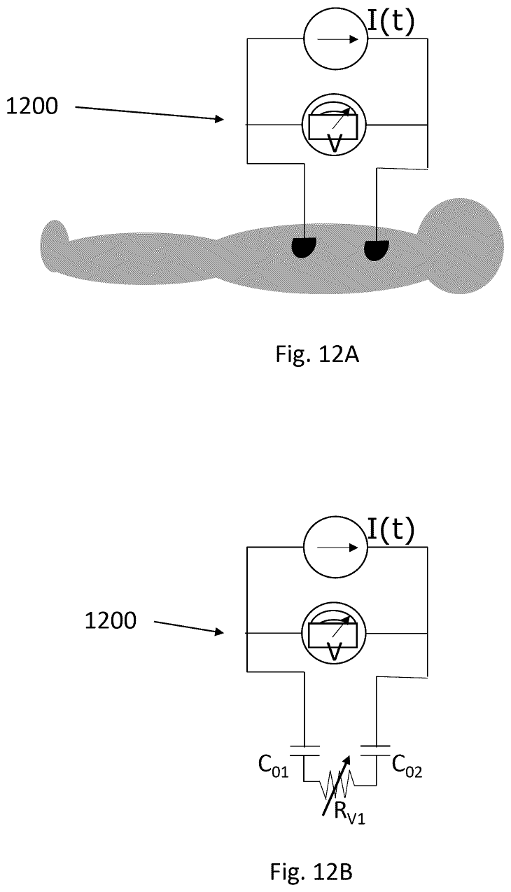

### Description Of FIGS. 12A-20B: Transthoracic Impedance Sensor

Alternatively, or additionally, a transthoracic impedance sensor may be used in some examples for the detection of the respiratory activity to determine the phase of a breathing cycle. This is discussed in connection with FIGS. 12A through 20B.

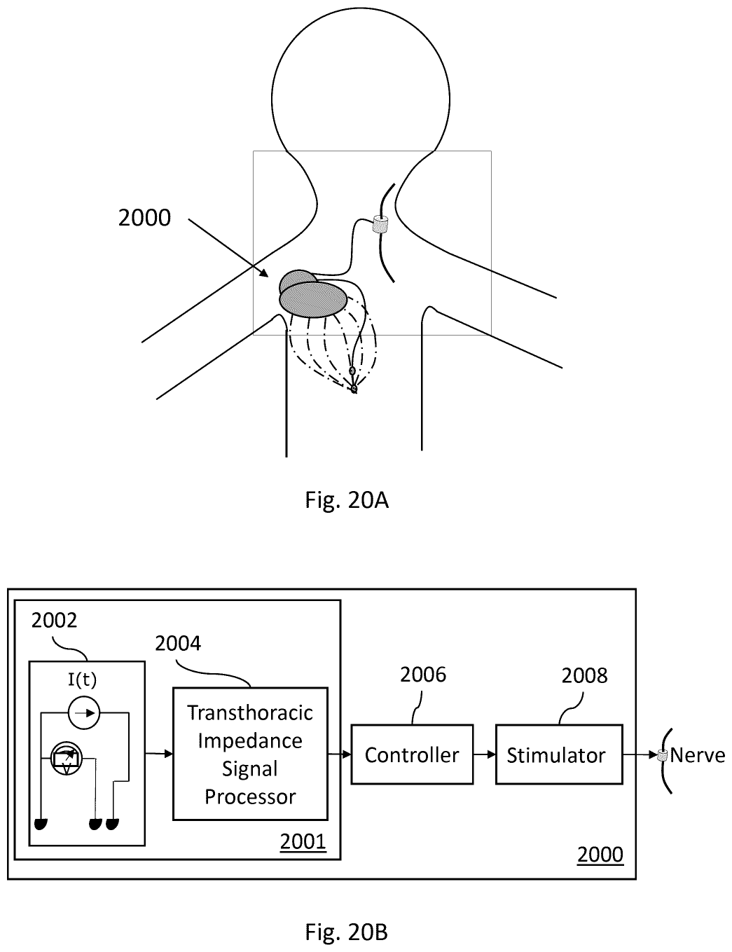

A transthoracic impedance sensor 2001 may be included in an example IPG 2000 (shown in FIG. 20B). The transthoracic impedance sensor 2001 can include a transthoracic impedance circuit 2002 and a transthoracic impedance signal processor 2004. The transthoracic impedance sensor 2001 is used by the controller 2006 to estimate the current phase of the respiratory cycle. Ultimately, the controller 2006 triggers a stimulator 2008 to generate the output pulses.

Different example transthoracic impedance circuits are discussed in connection with FIGS. 12A-17.

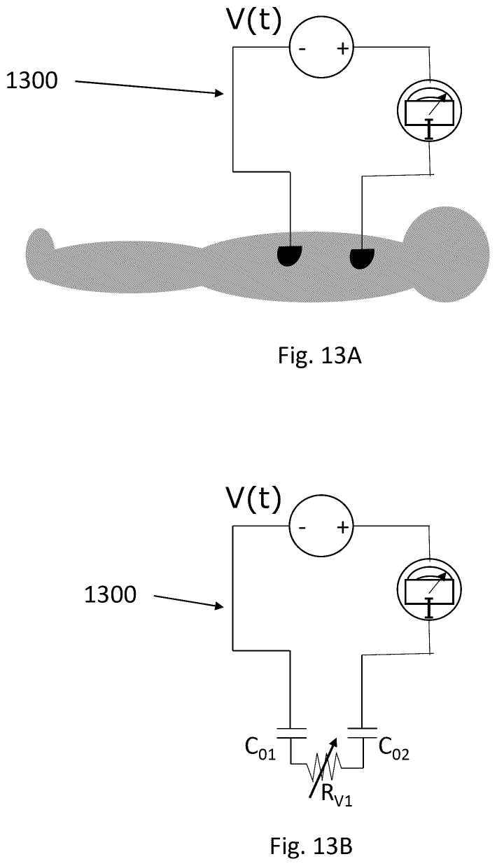

Turning to FIGS. 12A-13B, an example circuit that uses two electrodes is shown. The electrodes are positioned such that an increase in the amount of air in the lungs causes an increase in the electrical resistance that is observed between the electrodes. This illustrated example configuration may be referred to as a the bipolar configuration as there are two electrodes being used.

A transthoracic impedance measurement system using a bipolar circuit configuration is shown in FIG. 12A. In this case, the system applies a current waveform I(t) and monitors the resulting voltage waveform V(t). Transthoracic impedance is determined as the ratio of the voltage to the electrical current. FIG. 12B shows an electrical circuit model of a transthoracic impedance circuit 1200 where C₀₁ and C₀₂ are capacitors that represent the capacitances of the electrodes while R_{V1} represents the variable resistance due to respiratory activity. It should be noted that due to the application of the electrical current I(t) to the capacitors C₀₁ and C₀₂, there would be some voltage drop across those capacitors, which in turn would manifest in the measured voltage waveform V(t). Note that sure data may be ignored or not be desirable in some examples (e.g., a goal of the system is to measure the changes in R_{V1}).

Another implementation of the transthoracic impedance measurement system using a bipolar configuration is shown in FIG. 13A with transthoracic impedance circuit 1300. In this case, the system applies a voltage waveform, V(t), and monitors the resulting voltage waveform I(t). Transthoracic impedance is determined as the ratio of the voltage to the electrical current. FIG. 13B shows an electrical circuit model of the transthoracic impedance circuit 1300 where, once again, C₀₁ and C₀₂ are capacitors that represent the capacitances of the electrodes while R_{V1} represents the variable resistance due to respiratory activity. The measured electrical current would be different due to the presence of the capacitor, which in turn would somewhat interfere with the estimation of the transthoracic impedance, R_{V1}.

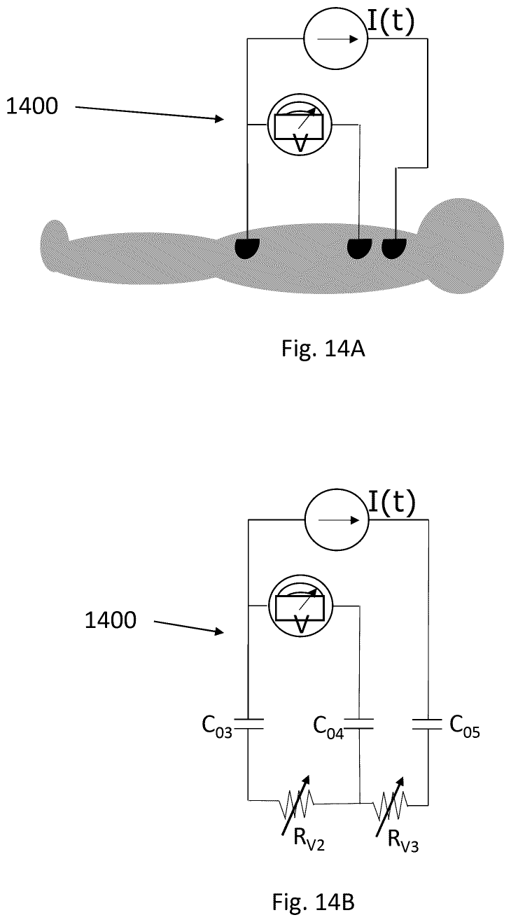

FIG. 14A shows the implementation of a transthoracic impedance measurement system using a tripolar system according to some embodiments. In this example, transthoracic impedance circuit 1400 includes three electrodes instead of only two electrodes as in the case of the bipolar systems shown in FIGS. 12A and 13A. In this configuration, the electrical current excitation and the voltage measurement parts of the circuit share only one electrode. It will be appreciated that “excitation” in this context refers to energization of the electrical circuit and creation of an electric field through body tissue (e.g., the thorax). This can be a difference from stimulation of a nerve or muscle tissue. In other words, the measuring of transthoracic impedance can be (and is) designed to be non-stimulatory. Therefore, the voltage drop across the capacitor C₀₅ of transthoracic impedance circuit 1400 becomes irrelevant as C₀₅ is no longer in the voltage measurement pathway. There is no voltage drop across C₀₄ as there is no current flowing over it. C₀₃ is common to the electrical current stimulation pathway and to the voltage measurement pathway, but it is possible to make C₀₃ large by using the metal case of the stimulator, hence minimizing the impact of the voltage drop across C₀₃. Thus, the tripolar implementation of transthoracic impedance circuit 1400 can be used to measure the transthoracic impedance, represented as R_{V2}, in a manner that may be more accurate than the bipolar implementation in some examples. Furthermore, the tripolar impedance measurement can be readily implemented in an implantable system by sending the electrical current from the tip electrode of a lead to the case of the device while measuring the resulting voltage drop between the ring electrode of the lead and the case of the implanted device.

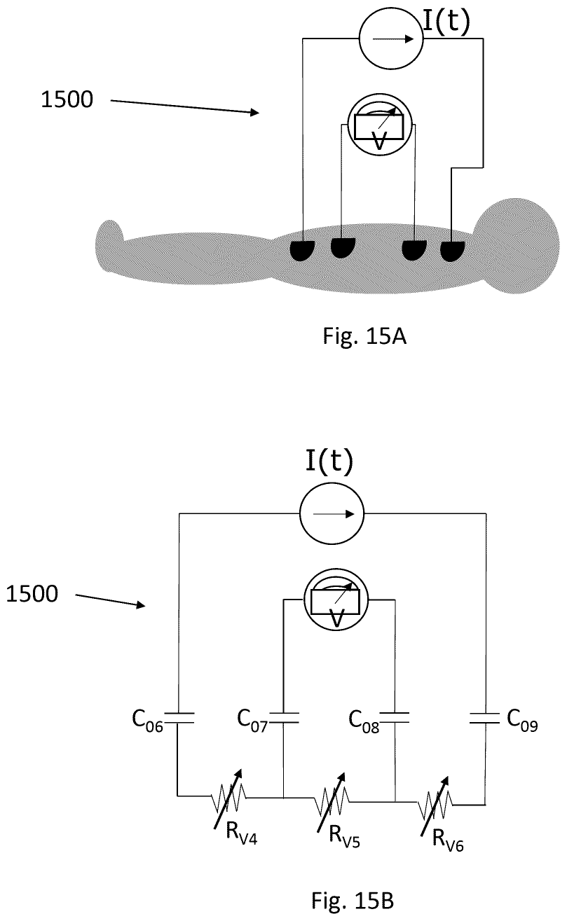

FIG. 15A shows the implementation of a transthoracic impedance measurement system according to some embodiments. In this example, transthoracic impedance circuit 1500 includes a quadripolar implementation comprising four electrodes, instead of only two electrodes as in the case of the bipolar systems or the three electrodes of the tripolar systems. In the quadripolar configuration, the electrical current excitation and the voltage measurement parts of the circuit do not share any electrodes. Therefore, the voltage drop across the capacitors C₀₆ and C₀₉ of FIG. 15B becomes irrelevant as neither C₀₆ or C₀₉ is in the voltage measurement pathway. There is no voltage drop across capacitors C₀₇ or C₀₈ as there is no current flowing over them. Therefore, the quadripolar impedance measurement with the transthoracic impedance circuit 1500 can be used to measure the transthoracic impedance, represented as R_{V2}, in a manner that may be more accurate than either the bipolar or tripolar implementations in some examples. However, compared to the tripolar configuration, the quadripolar configuration requires four electrodes, which may not be practical for all implantable systems.

Above discussions regarding FIGS. 12A-15B are focused on circuits that use a transthoracic impedance sensing lead that may be separate than the lead used for the delivery of the stimulation to the target tissue, e.g. phrenic nerve.

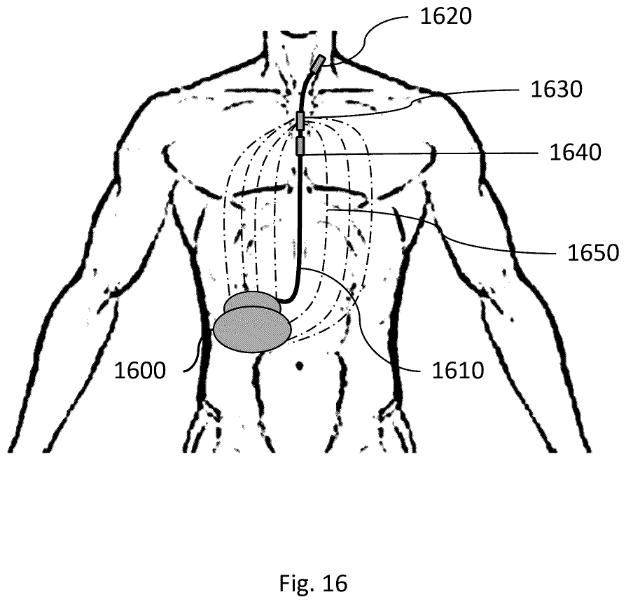

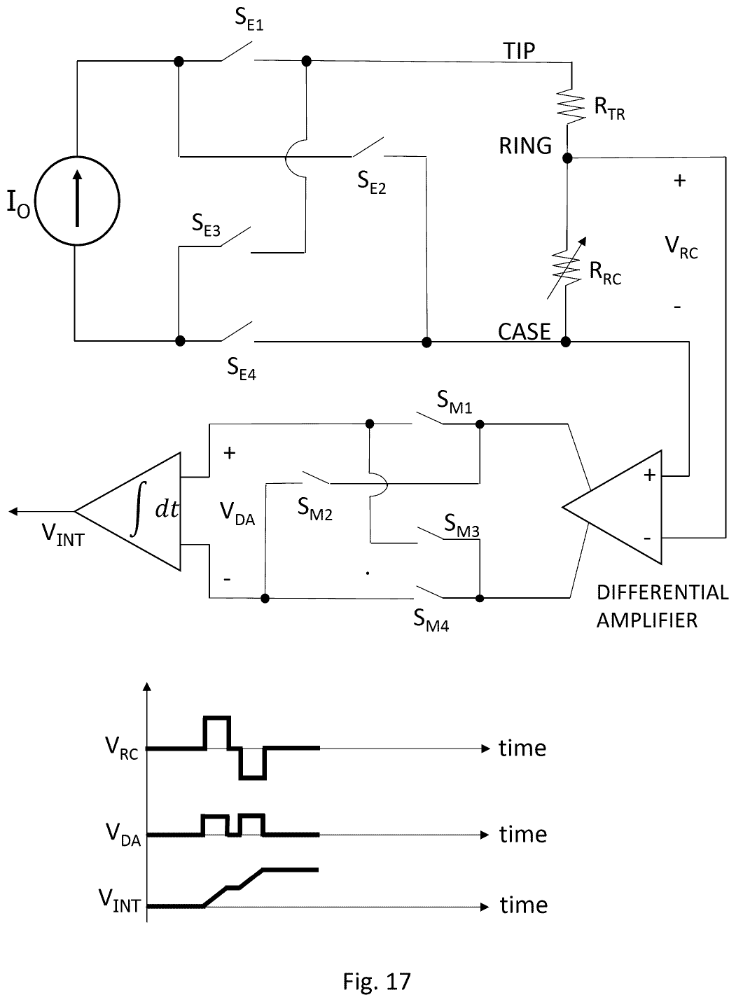

In some examples, the sensing lead and the lead that delivers stimulation may be the same. An example of such a configuration is shown in FIG. 16 where a single lead 1610 with multiple contacts (1620, 1630, 1640) used for both sensing of the transthoracic impedance and stimulating the target tissues. In the exemplary case illustrated in FIG. 16, an example stimulator 1600 is implanted in the abdominal area, and the single lead 1610 is tunneled under the skin, e.g., subcutaneously, to reach to the target nerve where the most distal contact 1620 is used for the delivery of the stimulation. Two additional electrical contacts (1630 and 1640) that are on the lead are located on the upper thoracic area and used for the measurement of the transthoracic impedance 1650 using the tripolar impedance measurement configuration formed by the contacts 1600, 1630, and 1640 as illustrated in FIG. 16.

In certain examples, measurement of the transthoracic impedance can present challenges for implementation in an implantable device. For example, there may be limited battery power for delivering excitation. Alternatively, or additionally, the measured signals can be relatively small and noisy for detection by the (relatively) limited electronics in an example implantable device.

FIG. 17 includes a diagram of an example implementation used in connection with a tripolar configuration. A current source I₀ is used to generate the excitation to be delivered to the tissue. To deliver a bi-phasic excitation, the direction of the electrical current is altered using the switches S_{E1}, S_{E2}, S_{E3}, and S_{E4}. When S_{E1} and S_{E4} are closed, the excitation current flows in forward direction across the chest resistors, R_{TR} and R_{RC}. When S_{E2} and S_{E3} are closed, the excitation current flows in reverse direction across the chest resistors, R_{TR} and R_{RC}.

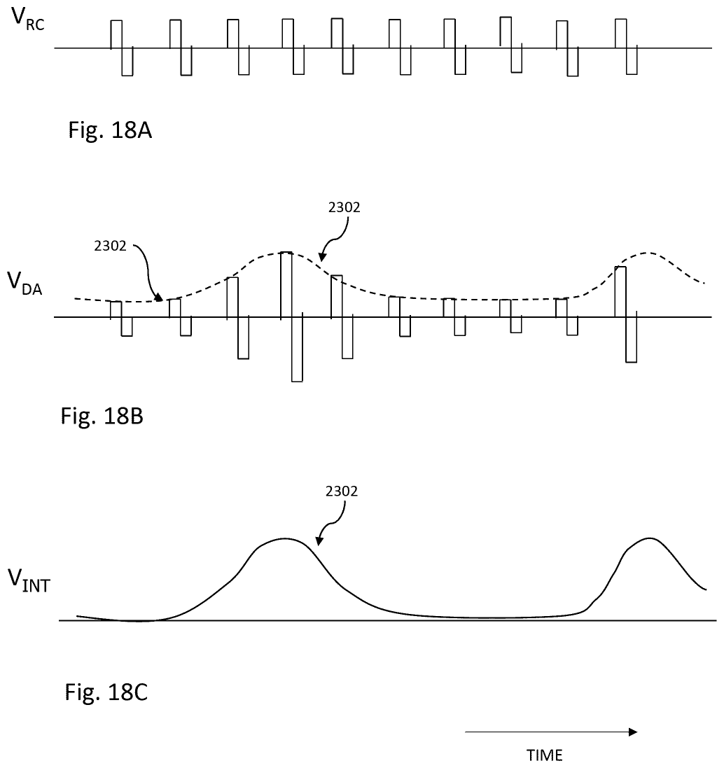

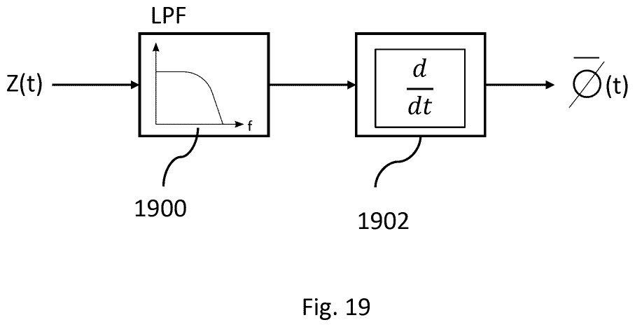

Repetition of this process results in the formation of the waveform shown as positive and negative pulses in FIG. 18A. Voltage drop between the ring and case, V_{RC} (FIG. 17), is measured using a differential amplifier and rectified using the switches S_{M1}, S_{M2}, S_{M3}, and S_{M4} which work in synchrony with the switches S_{E1}, S_{E2}, S_{E3}, and S_{E4}. Finally, the rectified signal V_{DA} is integrated to obtain V_{INT} which is proportional to R_{RC}.

A sample waveforms for V_{RC} and V_{INT} are shown in FIGS. 18B and 18C respectively. The vertical axes in FIGS. 18B and 18C are V_{rc} and Vᵢₙₜ respectively, and the horizontal axis is time. V_{INT} in FIG. 18C is taken as the transthoracic impedance signal 2302.

By its nature, the transthoracic impedance signal 2302 contains some noise due to, for example, motion artifacts, electrical interference, and/or Johnson noise that is present in electrical systems. Hence, in accordance with certain example embodiments, a low pass filter can be applied (at 1900 in FIG. 19) to a transthoracic impedance signal before further processing it, as illustrated in FIG. 19 to thereby generate an estimation of respiratory flow from (e.g., based on) transthoracic impedance. Afterwards, the transthoracic impedance signal 2302 may be differentiated (at 1902) to obtain an estimate of the flow signal, ϕ(t), which, in turn, can be used for the determination of the phase of the respiration, such as inhalation/ exhalation to be used by a controller of the implantable device for timing the stimulation to be delivered.

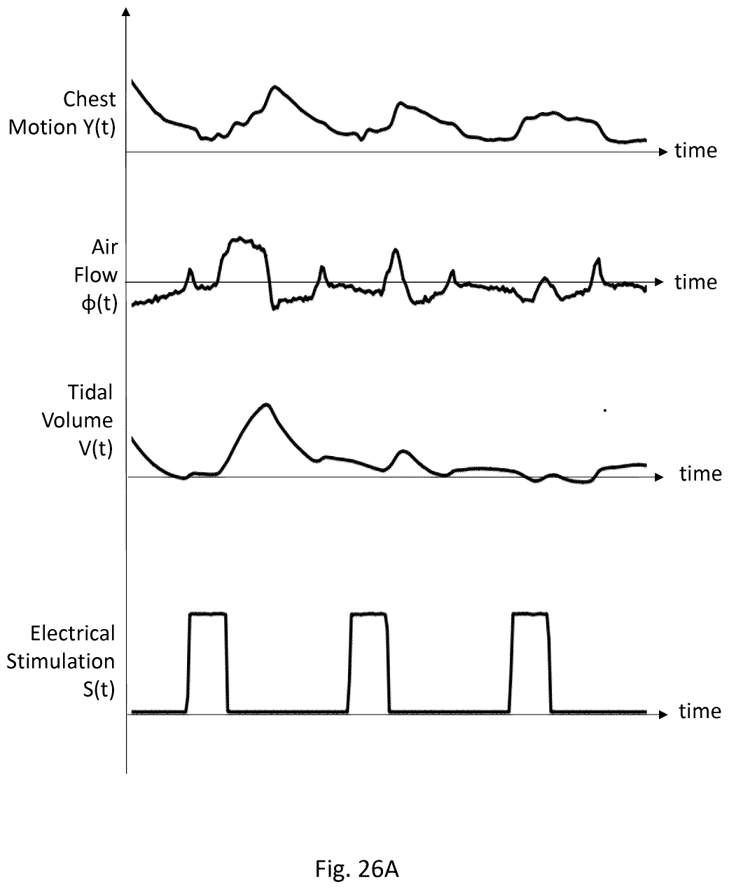

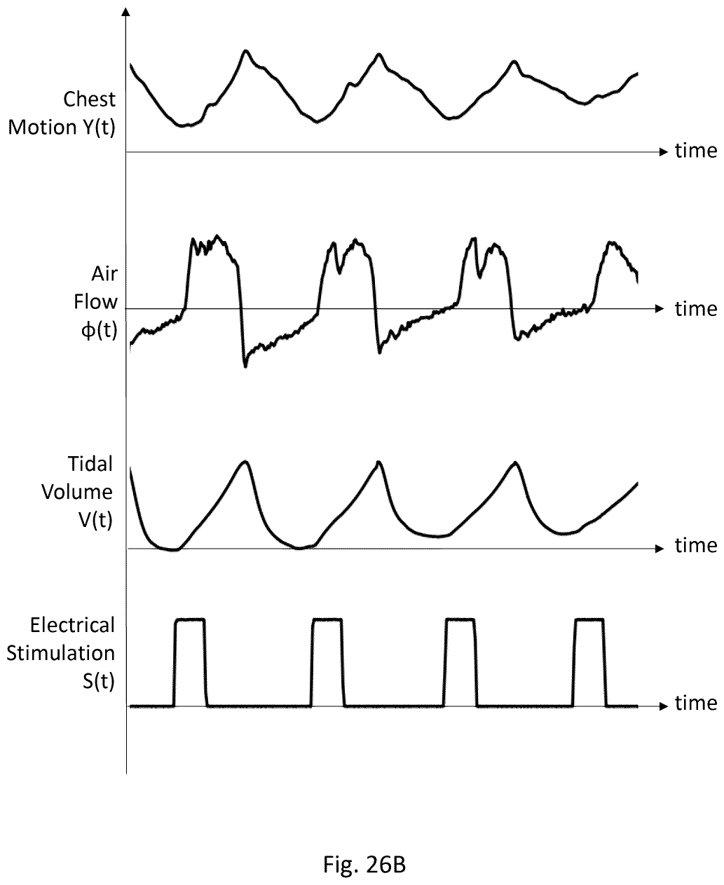

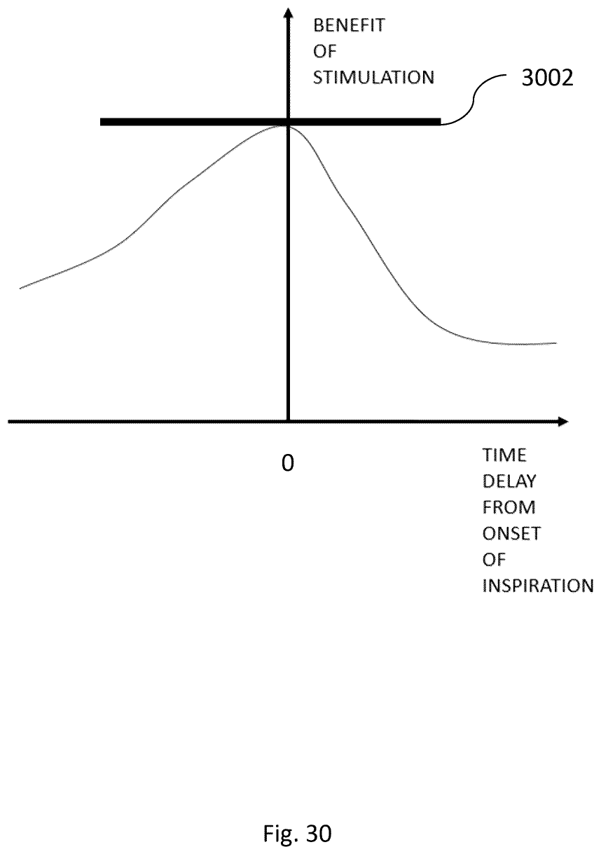

An exemplary implantable IPG 2000 using a transthoracic impedance based sensor is illustrated in FIG. 20A, where separate stimulation and sensing leads are used. A high-level block diagram of the system is shown in FIG. 20B. Briefly, a tripolar transthoracic impedance measurement circuit 2002 is formed using the tip and ring electrodes of the sense lead along with the case of the implantable stimulator 2000—e.g., as illustrated in FIG. 14A. A Transthoracic Impedance Signal Processor 2004—examples of which being illustrated in connection with FIGS. 17, 18A-18C and 19, produces an estimate of the respiratory signal, as shown in FIG. 18C. An increase in the transthoracic impedance, as shown in FIGS. 18 and 26, would increase additional air presence in the lungs resulting from the inhalation of air. Conversely, a decrease in the transthoracic impedance, again illustrated in FIGS. 18 and 26, would imply the exhalation. Hence, one can use the transthoracic impedance signal to impute the phase of the respiration, e.g., increases in the transthoracic signal corresponding the inhalation and decreases in the transthoracic signal corresponding the exhalation. The phase of respiration can be used by the controller to trigger the stimulation of the target nerve with proper delay, as shown in FIG. 30 and will be explained later.

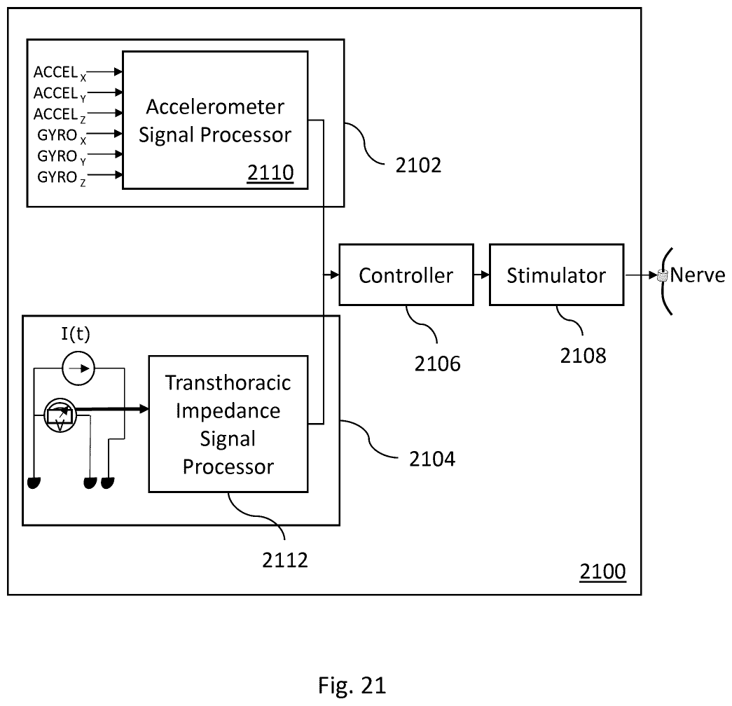

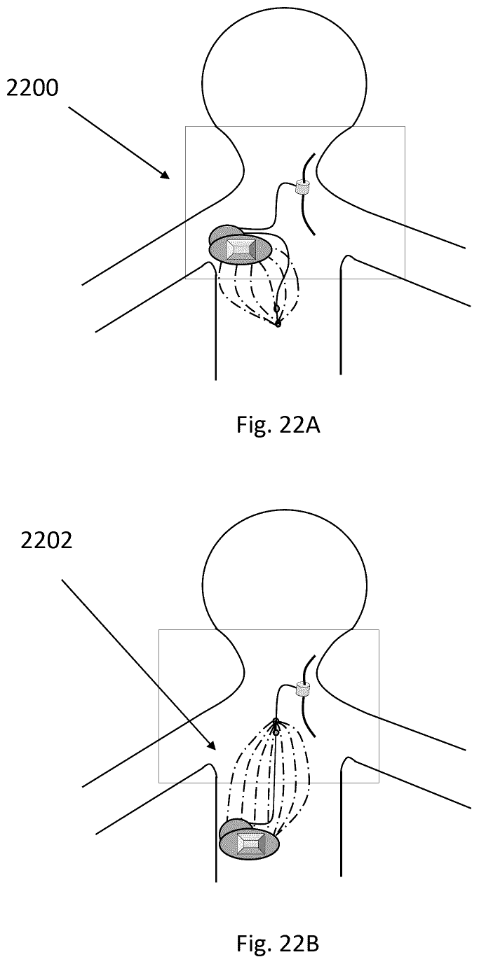

### Description Of FIG. 21-22B: Dual Sensor IPG

FIG. 21 is another block diagram of an implantable stimulator 2100 using a pair of sensors, namely an accelerometer sensor 2102 (which may be any of the accelerometer sensors described herein) and transthoracic impedance sensor 2104 (which may be any of the transthoracic impedance sensors described herein).

In some examples, the Controller 2106 receives inputs from both the Accelerometer Signal Processor 2110 and the Transthoracic Impedance Signal Processor 2112. The input are processed and the controlled causes the stimulator 2108 to stimulate the nerve of the patient.

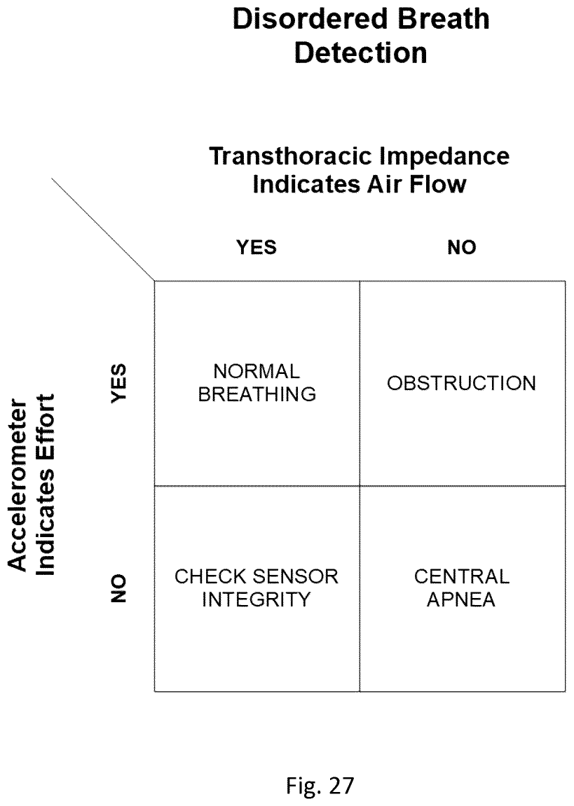

It will be appreciated that this type of implementation (e.g., with more than 1 sensor type) can provide for redundancy of sensory signals. Moreover, it may also allow the extraction of additional information from the respiratory waveforms. Additional details of such additional information is discussed in connection with FIG. 27.

FIG. 22A shows another example embodiment of the implantable stimulator 2200 using a pair of sensors, namely the accelerometers and transthoracic impedance sensor, as illustrated in FIG. 21. In this particular embodiment, the accelerometer is internal to the implantable stimulator and the transthoracic impedance measurement is provided with a dedicated lead that is distinct from the lead that is used for the delivery of the electrical stimulation to the target organ.

FIG. 22B shows another example embodiment of the implantable stimulator 2202 using a pair of sensors, namely the accelerometers and transthoracic impedance sensor, which was illustrated in FIG. 21. In this particular embodiment, the accelerometer is internal to the implantable stimulator, but the transthoracic impedance measurement is done with a combined lead that is used for the delivery of the electrical stimulation to the target organ and for the measurement of the transthoracic impedance.

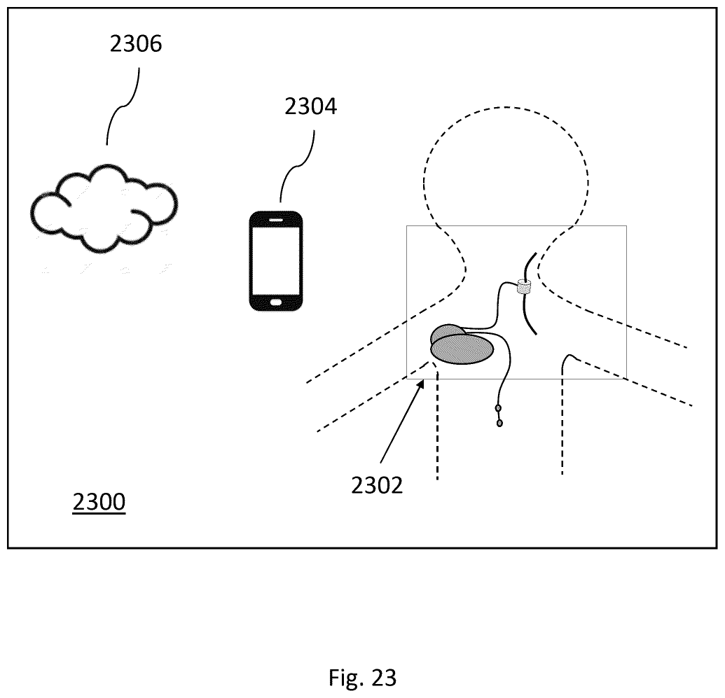

### Description Of FIG. 23: Example Medical System

FIG. 23 shows a diagram of a medical system 2300 that includes an implanted pulse generator 2302, stimulation delivery lead, transthoracic impedance sensing lead, a programmer (2304), which may be a mobile device. Also shown are remote computing resources that may be provided in a cloud-based computing environment. The system 2300 may also include, in addition to or as an alternative to transthoracic impedance sensors, sensors including one or more over an accelerometer, a gyroscope, an auscultatory sensor, ultrasonic sensor, a pressure sensor, an optical sensor, a pulse oximeter or a chemical sensor.

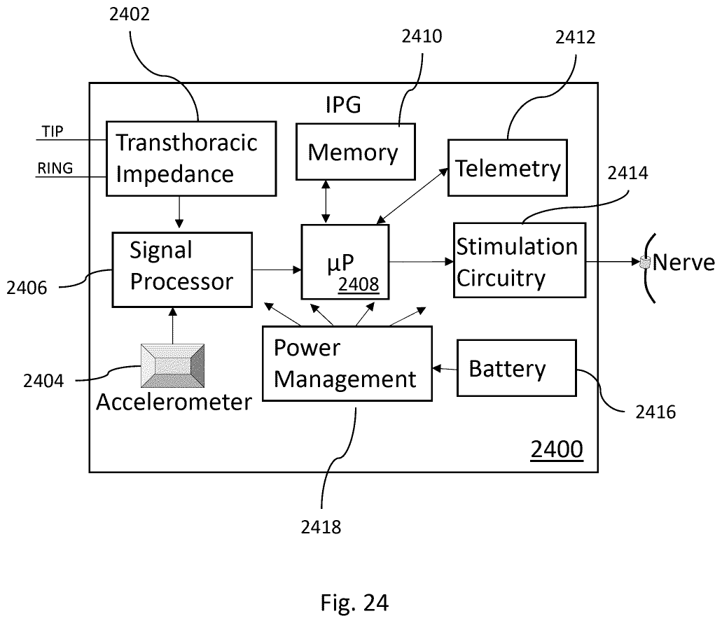

### Description Of FIG. 24: Example IPG

FIG. 24 shows a block diagram of an example implanted pulse generator (IPG) 2400, including the blocks containing the transthoracic impedance measurement subunit (2402), accelerometer subunit (2404), signal processor unit (2406) which may include an Accelerometer Signal Processor (e.g., 2110 or the like) and/or Transthoracic Impedance Signal Processor (e.g., 2112 or the like), Microprocessor (2408), Memory (2410), Telemetry (2412) which may include a wireless transceiver or the like, Stimulation Circuitry (2414), Battery (2416), and Power Management subunits (2418).

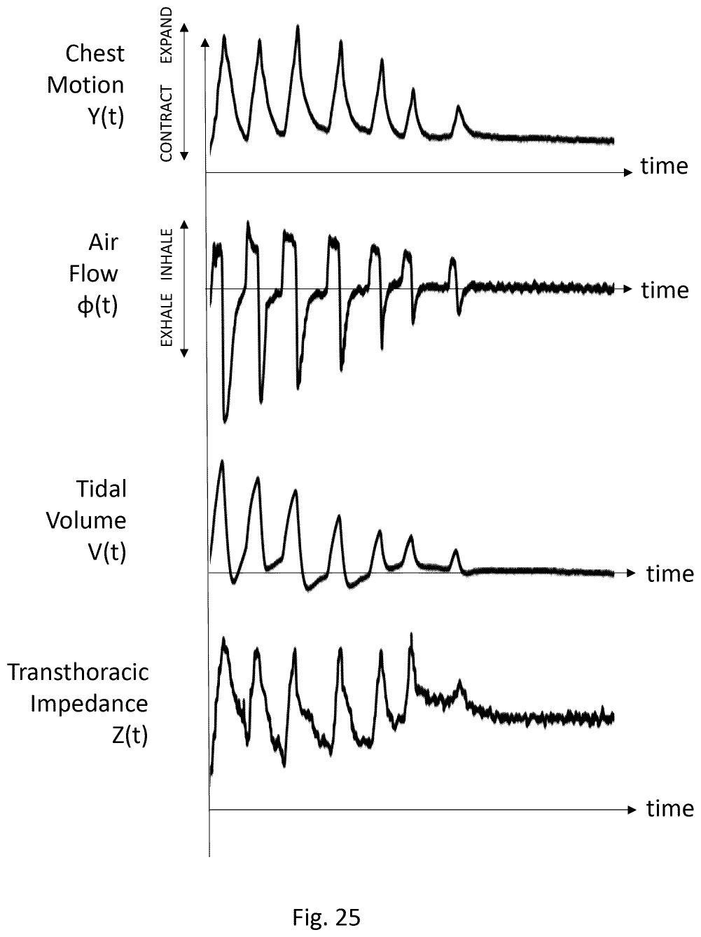

### Description of FIGS. 25-32

FIG. 25 shows recordings that were obtained as a patient transitioned from natural breathing to a central apnea in the absence of any therapy. As it can be seen from this trace, the patient took 4-5 normal breaths, and then the breaths got shallower, and eventually stopped breathing, as indicated by the diminishing amplitude of the Chest Motion, Y(t), Air Flow, ϕ(t) and Tidal Volume, V(t) traces. Transthoracic Impedance Trace closely follows the respiration signal indicating that transthoracic impedance can be used to determine the phases of the respiration, such as inhalation and exhalation as well as no air flow condition.

FIG. 26A shows the recordings obtained based on a patient receiving therapy in the form of electrical stimulation of the phrenic nerve. As shown in FIG. 26A, the first stimulation facilitated the patient taking in a deep breath (e.g. increased air flow and tidal volume), the stimuli were not as effective. Such ineffectiveness may be due to the not having the rate and/or phase of stimulation being matched to the respiratory cycle of the patient. Thus, in certain example embodiments, the timing of the stimulations in the respiratory cycle may be a factor in determining effectiveness of stimulation to increase air into lungs. In other words, synchronizing the stimulation to the.5 respiratory cycle and timing the stimulation to occur at a selected portion of the cycle (phasing) can be important to the effectiveness in applying nerve stimulation signals to increase airflow into lung(s).

FIG. 26B charts a series of breaths where the stimulation (S(t)) was synchronized and phased with chest motion (Y(t)) to cause deep inhalations (φ(t), V(t)). In other words, the stimulation rate matched (or was based on) the breathing rate of the patient and the simulation phase was matched to the respiratory cycle of the patient. Stimulation of a nerve, such as the phrenic nerve, that result in deep inhalations are effective to treat apnea.

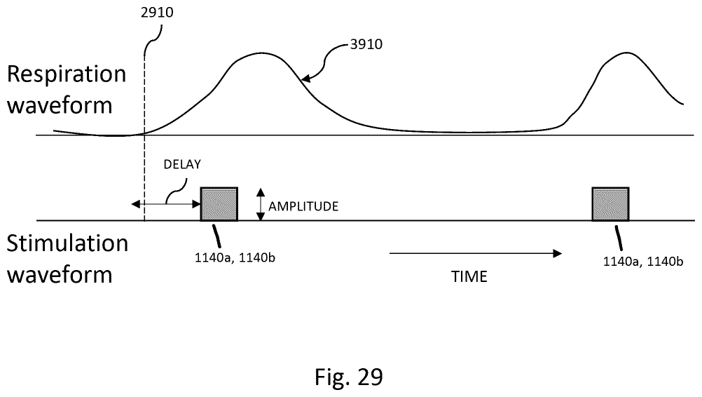

FIG. 29 shows the timing of the electrical stimulation in relation to the phase of the respiratory waveform. However, before going into the details of the timing of the stimulation waveform, first the parameters of the stimulation waveform (which may be called stimulation parameters herein) will be introduced.

Characteristics of the electrical stimulation waveform that is delivered to the target organ, such as the phrenic nerve, are shown in FIGS. 28A and 28B.

The waveform that is shown in FIG. 28A is monophasic, meaning that it only has a single polarity, positive in this case, while the waveform shown in FIG. 28B is biphasic, meaning that it has both positive and negative phases. Stimulation can be applied in the form of constant voltage or constant current. In some examples, constant current may be preferred for the stimulation on nervous tissue. Stimulation is usually delivered in the form of pulse trains 1140a/1110b (which is composed of one or more pulses 1112 or pulse pairs 1114), with a given amplitude 1120a/1120b, pulse width 1130a/1130b, and frequency 1110a/1110b (e.g., a period). Values chosen can be, but not limiting to 2 milli-Amps, 30 Hz, 250 micro-seconds, and lasting T_{TRAINS}=1 second. After a period of T_{TRAINS}, for example 4 seconds, another pulse train 1140a is delivered. A silent period (Tₛᵢₗₑₙₜ) 1145a, 1145b may separate each pulse train 1140a, 1140b. The silent period may be a few seconds (e.g., between 5 and 6 seconds), such as 2 seconds to 60 seconds, is adjustable and may be eliminated. All parameters of the stimulation waveform are adjustable and usually time dependent, meaning that the amplitude of the pulses may increase at the onset of the stimulation train, decrease toward the end. In some examples, frequency and/or the pulse width may stay constant or change throughout the stimulation train.

In some examples, stimulation pulses are delivered in bi-phasic pairs for active charge-balancing—this is shown at 1114 in FIG. 28B and more specifically in 2840 in FIG. 28C. Each pulse pair has a positive phase (2842) and a negative phase (2844), which may be separated by an Interphase Period (IP) 2846. Each pulse/pulse pair can thus be defined by Pulse Width 1130a/1130b and Pulse Amplitude 1120a/1120b. The pulse energy delivered by each pulse is thus based on both the positive and negative phase (e.g., and may be a sum or a product of the two). In certain examples the negative and positive pulses may be the same, except for the sign. In other examples, they may be different.

In some examples, stimulation bursts are a sequence of bi-phasic pulse pairs repeated at Frequency (F) for a Burst Duration. In some examples, such stimulation bursts may also include a burst rise time and/or burst fall time. Illustrative examples of such as provided in connection with 2850 in FIG. 28C.

In certain examples, the stimulation frequency can be around 30 Hz and may, in some examples, be within a range of 10 Hz to 140 Hz. In other words, the stimulation frequency may be related to the time period between individual stimulation bursts (e.g., 1110b). Accordingly, stimulation pulses of each stimulation burst may be delivered at a stimulation frequency (e.g., around 30 Hz) and the stimulation bursts may be delivered at a stimulation rate (e.g., that is based on the natural breathing rate of the patient).

In some examples, a stimulation pulse width can be about 300 micro-seconds and may, in some examples, be within a range of about 100 micro-seconds to about 1000 micro-seconds. In certain examples, longer pulse width may be based on the amplitude for a given pulse. In certain examples, longer pulse widths may be associated with decreased amplitudes while still providing effective respiratory therapy (e.g., which may cause less discomfort to a patient or less of a chance for the patient to wake up). In certain examples, an amplitude of the biphasic stimulation pulses (e.g., as shown in FIG. 28B), may be between about 0.5 to 1.5 milli-Amperes.

Stimulation example 2850 is an illustrative example of a bi-phasic stimulation burst 2852 that includes a burst rise time 2560, a plateau 2862, and a burst fall time 2864. Time periods that may be used for a burst rise time may vary between 0.2 seconds to 1.0 seconds—with certain examples being between 0.5 seconds and 0.9 seconds. Time periods that may be used for a plateau time may vary between 0.0 seconds and 2.0 seconds—with certain examples being between 0.3 seconds and 0.6 seconds. Time periods that may be used for a burst fall time may vary between 0.1 seconds and 0.5 seconds—with certain examples being between 0.1 seconds and 0.2 seconds.

In the example in FIG. 28c, a bi-phasic stimulation burst 2852 occurs over a time period of about 1 second. This includes a burst rise time 2560 of about 0.6 seconds, a burst fall time 2864 of about 0.1 seconds, and a plateau 2862 of about 0.3 seconds.

Over the course of stimulation burst 2852, multiple biphasic pulses are delivered. The pulse period 1110b (e.g., from the start of a first pulse to the start of a second pulse) can be between 30 and 40 (e.g., about 33.3) milliseconds (e.g., between 20 and 50 ms) with a pulse width 1130c (and/or 1130a/1130b) of about 300 microseconds (e.g., 305 μs in the example shown in FIG. 28C). The burst rise time 2560 starts from a first amplitude and gradually rises until reaching a threshold amplitude. The amplitude of the pulses (which may include multiple pulse-pairs) can remain constant over the course of 2862 (e.g., 0.3 seconds in FIG. 28C), until decreasing during burst fall time 2864. Note that the number of pulses included in the burst fall time 2864 may be 1. In other words, the burst fall time 2864 may start with the amplitude at the maximum (at the end of 2862), be decreased to an intermediate amplitude for a next burst, before turning off (e.g., at the start of 1145b). The rate of decease of the amplitude during 2864 may be greater than the rate of increase in amplitude during 2860. In certain examples, the number of bursts for which amplitude is increased is greater than the number of bursts for which amplitude is decreased. In certain examples, the amount of change in amplitude from successive bursts is less during 2860 than during 2864. In certain examples the number of bursts included in 2860 is greater than the number of bursts included in 2864. In certain examples the number of bursts in 2860 is greater than the number of bursts included in 2862.

In certain example embodiments, a stimulation burst (which may include multiple stimulation pulses) is shaped with a ramp up portion (which may correspond to 2860), a plateau portion (which may correspond to 2862), and a ramp down portion (e.g., which may correspond to 2864). The amplitude of each individual stimulation pulse within a stimulation burst may be delivered at an amplitude (e.g., in milliamps or other appropriate means for delivery of the targeted stimulation energy) defined by a ramp curve or ramp algorithm. Accordingly, the stimulation pulses (e.g., the amplitude thereof) may be selected to fit a ramp algorithm for delivery of a stimulation burst.

In some examples, a first stimulation pulse of a given stimulation burst may be delivered at a minimum threshold level rather than (for example) 0. In some examples, the minimum threshold level may be 0.2 milliamps, 0.25, 0.3, or the like. Successive increases may then be performed up until a plateau of between 0.5 milliamps and 1.5 milliamps. It was determined that smaller starting pulse values did not materially assist the patient in some instances. Accordingly, defining the pulse amplitudes of a pulse burst to start from a minimum threshold can beneficially provide power savings and/or decrease ineffectual electrical stimulation of the patient. In certain example embodiments, ramp increases and/or decreases may be linear or exponential in nature.

As shown in FIG. 29, the onset of the stimulation train 1140a, 1140b may occur a certain time period after (or in relation to) the onset of inhalation (2910) phase of the respiration waveform 3910, as denoted by the label “DELAY” in FIG. 29. DELAY could be a positive number, a negative number (e.g., before the onset of inhalation), or zero.

FIG. 30 shows an illustrative example of results (e.g., based on aggregate results from the studies done with human subjects) where the plot indicates that the benefit of the stimulation is a function of the “DELAY” time, where the benefit was defined as one or more of tidal volume, tissue oxygen saturation, flow rate, and total ventilation. However, it should be noted that the optimal stimulation DELAY may not be fixed, and there may be intra-patient and inter-patient variability. In other words, the local maximum of the benefit may be different for different patients, or the same patients. In an instance, the local maximum may be prior to the onset of inspiration. In another instance, the local maximum may be at the onset of inspiration. And in another instance, the local maximum may be after the onset of inspiration. Therefore, the Controller works to maximize the benefit, or the objective function, methods of which will be described next.

It should be noted that the vertical axis of the plot shown in FIG. 30 is labelled as “Benefit of Stimulation” which may be based on any or all of the following parameters (collectively referred to as breath characteristics): tissue oxygenation, oxygen desaturation index, apnea hypopnea index, ventilatory flow, tidal volume, minute ventilation and the respiratory duty cycle, which is also known as Ti/Ttot, refers to the ratio of inspiration time (Ti) to the total respiratory cycle time (Ttot) during a breath. These parameters can be derived from the data signals obtained from a patient—such as the transthoracic impedance, accelerometer, or the externally measured signals such as the pulse oximeter and the milli-meter radar. Such data points may be used to generate a Benefit of Stimulation metric that is used to determine when stimulation should be triggered with respect to inspiration by a patient. Furthermore, horizontal axis of the plot shown in FIG. 30 is labelled as “Time delay from onset of inspiration” corresponds the variable labeled as “DELAY” in FIG. 29

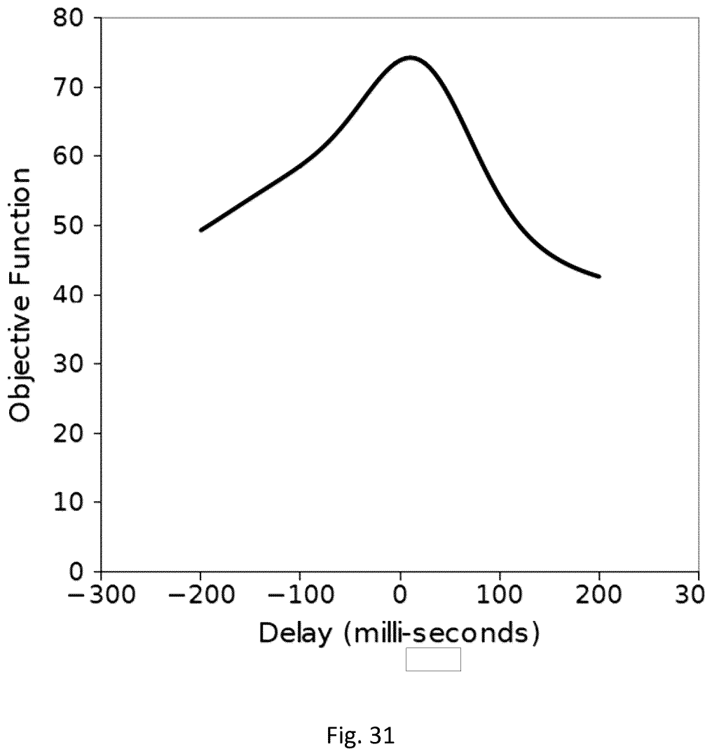

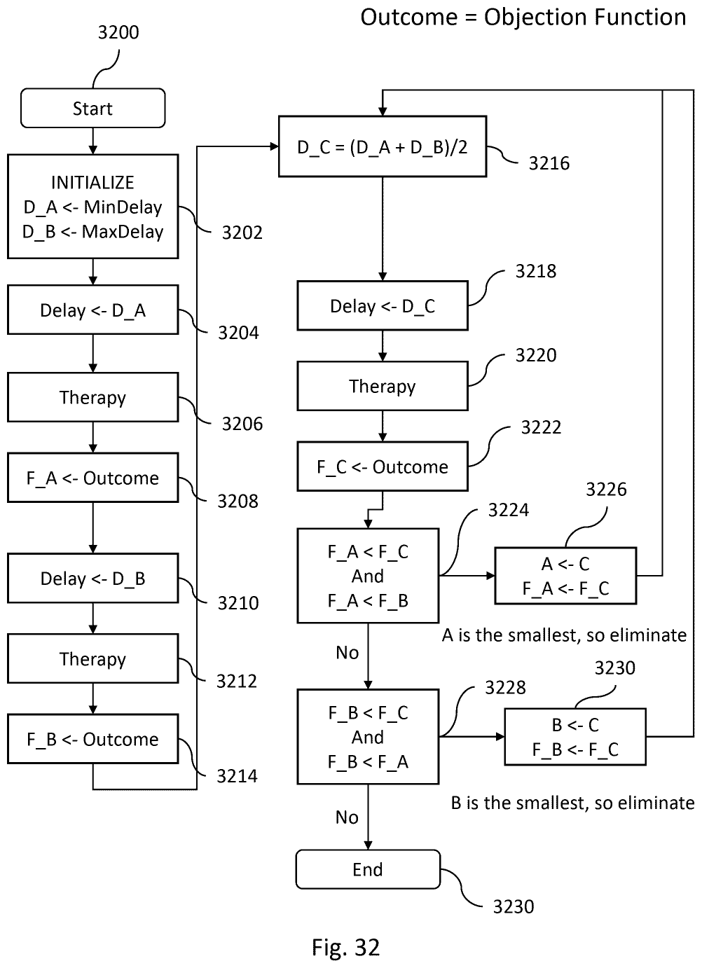

FIG. 31 illustrates the operation of an exemplary algorithm that may be used by the Controller. The algorithm is shown as a flowchart in FIG. 32. Although the morphology of the Objective Function, which is the benefit of the stimulation, is shown in FIG. 31, in other instances (e.g., under real-world/arbitrary conditions), the shape of the curve is not known and may change over time. Hence, the Controller periodically searches for the optimal operating point, or the best DELAY within a given window. At 3200, the algorithm beings and initializes, at 3202, minimum and maximum delay values. For the example shown in FIG. 31, algorithm picks two DELAY values, A=−150 milli-seconds and B=+120 milli-seconds. At 3216, a third value is chosen as the midpoint between A and B, which is C=−15 milli-seconds. All three DELAY values are attempted, and the resulting values of the objective function are recorded as F_A, F_B, and F_C.

Referring more specifically to FIG. 32, at 3204, the delay A value is set as the delay value to use for therapy at 3206. A resulting value from the outcome of that therapy is recorded as F_A at 3208.

Next, at 3210, the generated B delay value is set as the delay for therapy and therapy is performed at 3212. A resulting value from the outcome of that therapy is recorded as F_B at 3214.

Next, at 3218, the generated C delay value is set as the delay for therapy and therapy is performed at 3220. A resulting value from the outcome of that therapy is recorded as F_C at 3222.

The results are tested at 3224 and/or 3228. 3224 tests if the resulting outcome from Delay A is the smallest result (e.g., the smallest output from the objective function), if it is then A is eliminated by overwriting delay A with the generated C delay value at 3226. The process returns to 3216 where a new C value is generated using the newly assigned A value and the previously assigned B value.

If, at 3224, F_A is not the smallest, then the process continues by testing if F_B is the smallest at 3228. If F_B is the smallest, then B is overwritten with the values from previously generated C at 3230 and the process returns to 3216 to generate a new C value.

The process continues until neither A or B are the smallest (e.g., F_C is the smallest) before ending at 3230.

In the example that is shown in FIG. 31, the vertical axis could be the peak flow rate in Liters/minute, although other measurements, such as the tidal volume could be chosen as the objective function. At this point, the DELAY value giving the lowest benefit, which is B, is dropped and the process is repeated. The algorithm ends when F_A and F_B are both equal or greater than F_C. This may occur when F_A, F_B, and F_C are equal. By repeating, the algorithm effectively searches for a DELAY value that results in the greatest value of the function F. The above-mentioned algorithm determines an optimal value of the DELAY to cause increased airflow to lungs. It is envisioned that variations of the algorithms may be used by the Controller to determine the DELAY value. Other algorithms, such as Golden Section Search, Exhaustive Search, Newton's Method, AMEOBA (aka Downhill Simplex Method), can be used.

Based on the DELAY value that is determined to be the optimal one for a given patient under given conditions, stimulation may be delivered during late expiratory period, mid expiratory period, or early expiratory period, as well as the late inspiratory period, mid inspiratory period or early inspiratory period. Furthermore, the determination of the optimum stimulation delay could be done for the maximization of one or more breath characteristics, including but not limited to tidal volume, air flow and airway patency.

It should be noted that the therapy is applicable for the treatment of multiple forms of sleep disordered breathing, including obstructive sleep apnea (OSA), central sleep apnea (CSA) as well as mixed apnea and hypopneas.

The advantage of the use of sensors to determine the respiratory phase to adjust the timing of the stimulation was illustrated by the graph that is shown in FIG. 29.

Although the invention covered the use of two specific sensors, namely the transthoracic impedance sensor and the accelerometers, other transducers, such as auscultatory sensors (microphones), ultrasonic sensors, pressure sensors, tissue oxygen sensors and chemical sensors could also be used for similar purposes. As mentioned earlier, use of multiple sensors not only provides the benefit of redundancy in the case of a sensor failure, but also provides additional information that may not be available in a single sensor system. This is shown by the table in FIG. 27.

A transthoracic impedance signal can be used for the confirmation of the air flow, while the accelerometer signal can be used for the confirmation of the patient's effort to breathe. In such instances, the following conclusions can be made by observing the sensor outcomes, as shown in FIG. 27:
Transthoracic Impedance indicates Air Flow, and
Accelerometer indicates Presence of Effort, then
the conclusion is successful breath.
Transthoracic Impedance indicates No or Low Air Flow, and
Accelerometer indicates Presence of Effort, then
the conclusion is Obstructed Air Flow (OSA).
Transthoracic Impedance indicates No or Low Air Flow, and
Accelerometer indicates Absence of Effort, then
the conclusion is Central Apnea (CSA).
Transthoracic Impedance indicates Air Flow, and
Accelerometer indicates Absence of Effort, then
the conclusion is Conflicting Sensor Data.

It should be noted that the data presented in FIGS. 25, 26A and 26B were from a single patient and examples given were for specific cases, the conclusions remain general, and the invention encompasses embodiments similar to those described above.

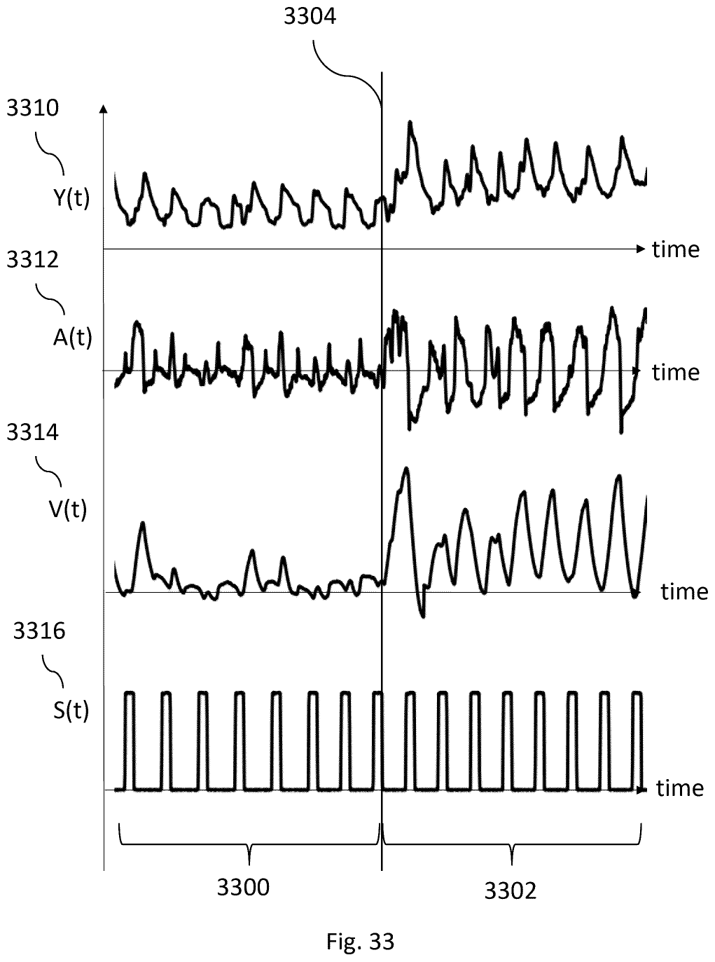

### Description Of FIGS. 33-37: Entrainment

During runtime of an implantable device that is delivering stimulation to a patient it is possible to run it in an entrainment mode. This is described below. In certain examples, various techniques for controlling a stimulator may be performed such that the controller may adjust one or more stimulation parameters (also called characteristics of stimulation energy in some examples) used in delivering stimulation energy to a nerve (e.g., the phrenic nerve or other nerve as discussed herein). Stimulation parameters as discussed in connection with, among other places, FIG. 28A and 28B and can include: stimulation rate, stimulation phase, pulse amplitude (e.g., amplitude), pulse width, frequency, burst duration, burst rise time, burst fall time, interphase period, and the like.

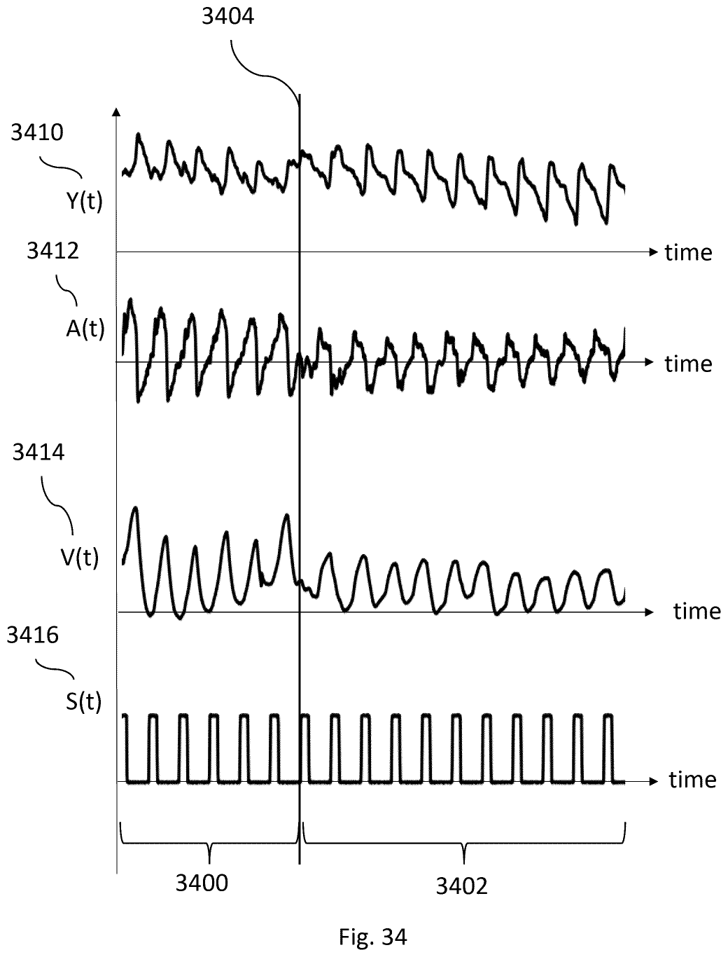

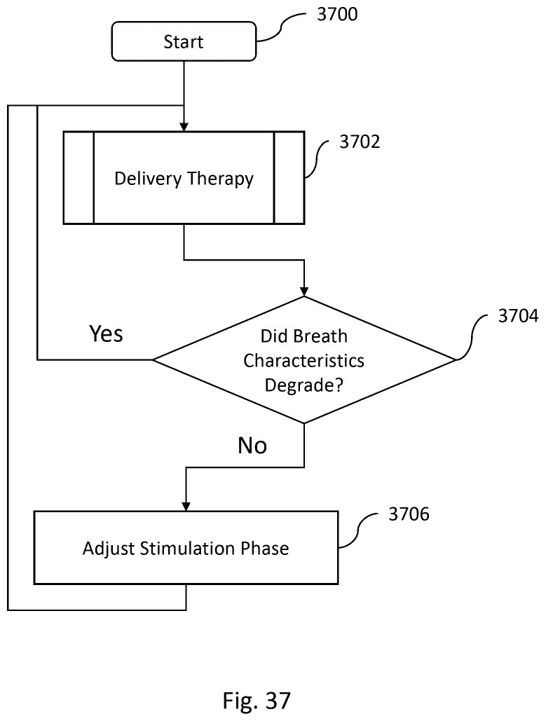

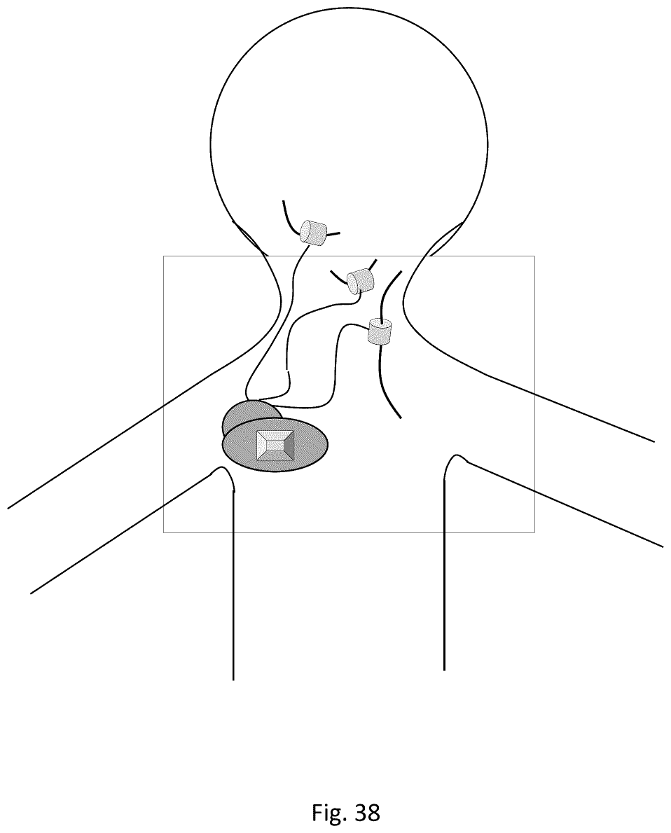

In some examples, a rate of stimulation may be determined and controlled. Example techniques for controlling the rate of stimulation (also called a stimulation rate or rate herein) are shown in FIGS. 33, 35, and 36 and additional details regarding determining or controlling the rate are discussed in connection with FIGS. 28A-28B, for example. In some examples, a phase at which stimulation is performed (also called a stimulation phase or phase) by the stimulator is determined and controlled. This is shown in FIGS. 34 and 37, with additional details regarding determining or controlling the stimulation phase discussed in connection with FIGS. 28A-32, for example.

FIG. 33 includes 4 graphs with chest motion Y(t) 3310, air flow A(t) 3312, and tidal volume V(t) 3314 each representing different breath characteristics of a patient. Electrical stim S(t) 3316 represents the stimulation parameters used in connection with the delivery of stimulation energy to the patient (e.g., via IPG). In this example, the stimulation rate is adjusted. Specifically, FIG. 33 illustrates start of entrainment as the stimulation rate is increased. During this period the stimulation rate can be increased by reducing, for example, the TSILENT parameter that is described in connection with FIG. 28.

As shown in FIG. 33, the first eight stimulation trains that were delivered did not cause entrainment. More specifically, a less-than-optimal airflow and tidal volume where generated—as shown in the signals on the left half 3300 the panel in FIG. 33. In some examples, based on determination that the airflow and/or the tidal volume is insufficient (e.g., as represented in FIG. 33), the stimulation rate may be increased at 3304. The determination to increase the stimulation may be made by an IPG described herein or other component of medical system as described herein. In some examples, the determination is made by a clinician and in other examples the determination is dynamically performed (e.g., by an IPG).

In the stimulation trains shown on the right half 3302 of FIG. 33 the stimulation is increased. For example, the T_{SILENT} period between trains is reduced in 3302 as compared to the left half 3300 trains. The right half 3302 pulse trains achieved entrainment in which the respiration pattern of patient was in synchrony with the electrical stimulation trains resulting in significant increases of air flow and tidal volume.

Monitoring of a patient may continue during entrainment. Monitoring can be used to determine if entrainment loses efficacy (e.g., if the efficacy of therapy drops below a given threshold). An example of this is shown in in FIG. 34. The graphs in FIG. 34 include chest motion Y(t) 3410, air flow A(t) 3412, and tidal volume V(t) 3414 that each represent one of different types of breath characteristics of a patient. Electrical stim S(t) 3416 represents the stimulation parameters used in connection with the delivery of stimulation energy to the patient (e.g., via IPG). In this case, the stimulation rate (e.g., as determined based on FIG. 33 above) was constant throughout the trace that is shown in FIG. 34. The phase of the respiratory cycle of the patient was in synchrony with the stimulation trains being delivered for the first six cycles of period 3400. During this period, but after the 6^{th} cycle, a phase shift (at 3404) between the respiration of the patient and the stimulation trains developed. Even though the respiratory rate of the patient was same as the stimulation rate, the presence of a phase shift at 3404 caused a significant loss of efficacy of as indicated by the reduced air flow and tidal volume as indicated over period 3402. In this situation, a search for the optimal stimulation DELAY can be triggered. In some examples, the algorithm that is executed may be the same or similar to the one that is illustrated in FIGS. 31 and 32.

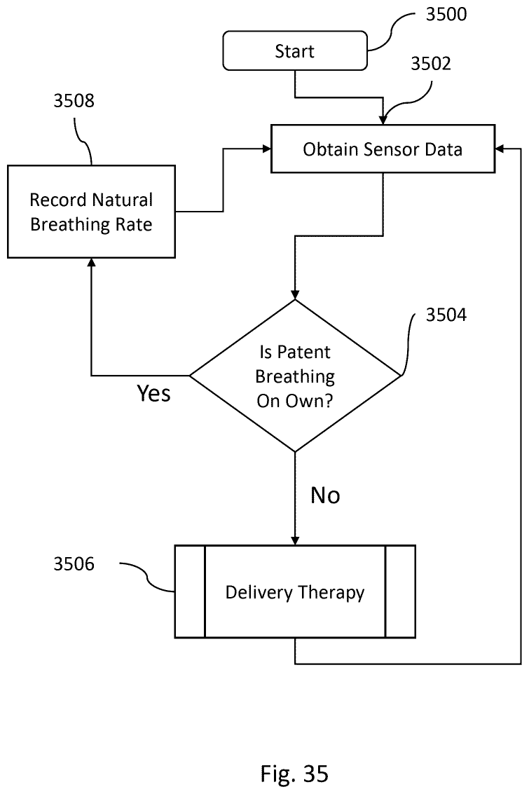

FIG. 35 shows a flowchart of the algorithm used before entrainment. The purpose of this algorithm shown in FIG. 35 is to determine the natural breathing rate of the patient and also capture the characteristics of the natural breathing. In some cases, this may include determining/capturing the awake breathing pattern of the patient. At 3500, the process starts and at 3502, sensor data from the patient is obtained (e.g., as described in connection with FIGS. 9-22B). At 3504, a determination is made as to whether the patient is breathing on their own. This determination may be based on the sensor data from 3502 and/or may be based on, for example, clinician observation. In some examples, the determination may be made by a controller of an IPG (or other associated controller). In other examples, the determination may be performed by a clinician. If the patient is not determining to not be adequately breathing, then therapy is delivered at 3506 and the process loops back to 3502/3504. If the patient is breathing on their own, then the natural breathing rate of the patient is recorded at 3508. In some examples this is stored to memory of the IPG and in other example this information may be communicated to other processing resources in communication with the IPG. In any event, once the natural breathing rate is recorded, the process returns to 3502/3504. The delivery of therapy may include the processing shown in FIGS. 32, 36, and 37 according to certain examples. In other words, once the patient is determined to have stopped breathing (or is having trouble doing so), either due to a central or obstructive apnea, then the therapy processing is initiated as is discussed herein.

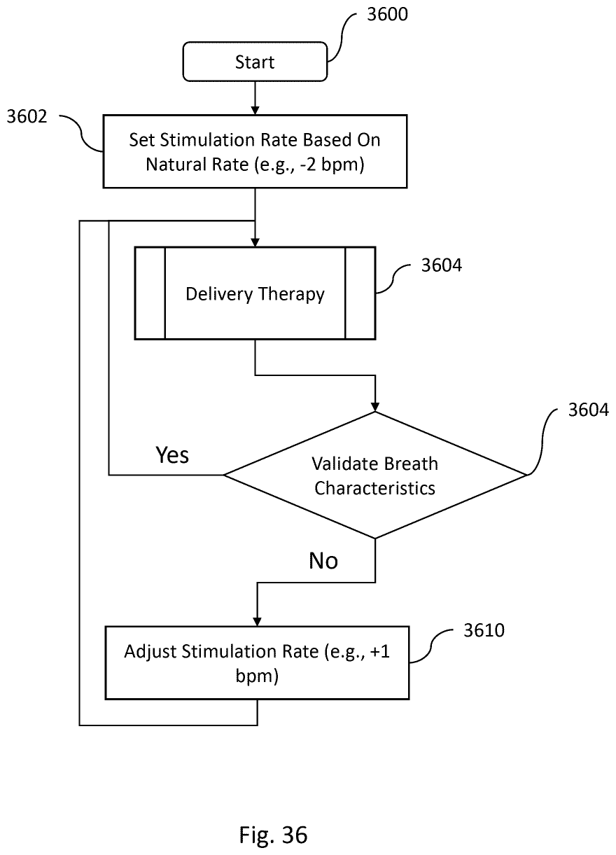

FIG. 36 shows a flowchart of the algorithm used to initiate the entrainment. The process shown in FIG. 36 may be used to generate a stimulation rate that matches (is linked to or based on) the natural breathing rate of the patient (e.g., as determined at 3508). In certain example embodiments, the stimulation rate is the number of times a stimulation burst, or stimulation train is delivered. For example, the number of times stimulation burst 2852 is delivered in a period of time (e.g., one minute). The process starts at 3600. At 3602, the natural rate for the patient is retrieved and the stimulation is delivered at a rate based on the stimulation. In this example, the stimulation rate is initially set to be 2 breaths per minute less than the natural respiratory rate of the patient. With the rate set, therapy may be delivered at 3604. As part of therapy, sensor data of the patient may be obtained to determine the breath characteristics of the patient. If the breath characteristics are valid, the process returns to 3604. If the breath characteristics indicate a problem (e.g., the breath characteristics are below a given threshold or the like), then, at 3610, the stimulation rate may be increased. In some examples, increases in stimulation rate can be performed in small (e.g., +1 or +2 per minute) steps. After each increase, breath characteristics of the patient may be calculated again, as shown in FIG. 36. If entrainment is achieved (e.g., based on the breath characteristics at 3604), then the stimulation continues at the new stimulation rate. Otherwise, the stimulation rate is increased further, and the cycle repeats as illustrated by the flowchart shown in FIG. 36. Once the proper stimulation rate is established using the algorithm shown in FIG. 36 (e.g., based on the breath characteristics), the phase shift value may be determined using the algorithms discussed in FIGS. 32 & 37. In some example embodiments, the rate can be increased until the entrainment is achieved.

FIG. 37 shows a flowchart of the algorithm used to maintain the entrainment and to ensure that a new DELAY value (which may represent the phase at which stimulation is delivered) is calculated when a phase shift develops between the respiratory rate of the patient and the stimulation rate.

At 3700, the process is triggered, and therapy is delivered at 3702. At 3704 a determination is made as to the breath characteristics. For example, the process determines whether the breath characteristics of the patient have fallen below a given threshold. If the breath characteristics remain elevated then the process returns to 3702 and therapy may continue to be delivered.

If, however, the breath characteristics have decreased below a given threshold, then the stimulation phase may be adjusted at 3706. This may trigger the processing shown in FIGS. 31-32. In some examples, the processing for adjusting the stimulation phase may be performed without also adjusting the stimulation rate. Indeed, the stimulation rate may be keep constant while the stimulation phase is adjusted.

In certain example embodiments, as discussed above, a rate of stimulation is matched against the rate of breathing by a patient. This rate may initially be lower than the breathing rate of the patient. However, the stimulation rate may be increased in certain examples. In some examples, this may be because the patient's breathing rate has increased, and in other examples this may be due to better responsiveness to stimulation therapy which a relatively higher stimulation rates (in relationship to breathing rates).

Once the stimulation rate is matched to the breathing rate, then the phase at which stimulation is to be delivered may be determined and/or set. Details of determining phase are discussed in connection with FIG. 32 and elsewhere. If the patient breathing adjusts such the stimulation is no longer in phase (e.g., based on feedback from sensors, etc.), then the phase at which stimulation is delivered may be adjusted.

Once the stimulation phase is matched to the breathing cycle of the patient, then the amplitude may be adjusted as needed. Examples of amplitude adjustment as shown in, for example, FIG. 6. For example, even though the stimulation rate and phase may be optimized for a given patient (e.g., the breathing efficacy is a local maximum, but is still insufficient for maintaining airway patency), the amplitude or other stimulation parameters may be adjusted. Accordingly, for example, stimulation rate and/or stimulation phase may be “locked” or the like while other stimulation parameters are adjusted.

The process of adjusting the rate, phase, and amplitude (or other stimulation parameters) may continue throughout a sleeping period for a patient. In other words, sometimes the rate may be adjusted, sometimes the phase may be adjusted, and other times the amplitude may be adjusted. Each of these adjustments may result in modifying how stimulation energy is applied to the nerve (such as the phrenic nerve) of the patient.

In certain example embodiments a therapy process is executed on one or more hardware processors (e.g., the IPG) that includes a detection process, an adjustment process, and an optimization process. The detection process includes detection of: 1) respiratory rate of the patient; 2) a phase difference between the respiratory cycle of the patient and the stimulation cycle, and 3) breath characteristics of the patient. The adjustment process includes adjusting, based on the data detected from the detection process, or controlling: 1) the rate of stimulation, 2) the phase at which stimulation is delivered, and 3) characteristics of the stimulation burst (and or pulses thereof) that include frequency, amplitude, pulse width, ramp up duration, and ramp down. In some examples an optimization process may be used to determine how the adjustment process modifies aspects at which the stimulation energy is delivered. In some examples, the optimization process uses breath characteristics such as the flow rate and the tidal volume. In some examples, the optimization process includes executing a search algorithm(s) to determine the necessary stimulation rate and/or phase. Illustrative example search algorithms include Golden Section Search, Exhaustive Search, Newton's Method, AMEOBA (aka Downhill Simplex).

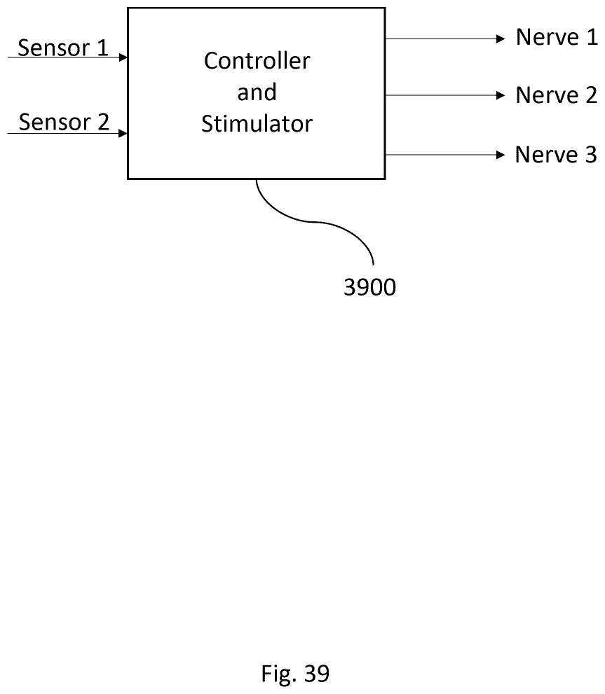

### Description Of FIGS. 39-42: Multi-Target

Although the discussions to up to this point referred to a stimulator that is delivering stimulation to a single nerve such as a phrenic nerve, an embodiment of the invention delivers stimulation to multiple targets simultaneously or selectively as illustrated in FIG. 39. Example nerves that are targeted include, but not limited to, phrenic nerve, hypoglossal nerve, the ansa cervicalis, and others.

FIG. 39 shows a high-level block diagram of a multi-target stimulator 3900. It accepts inputs from one or more sensors (or two or more sensors), such as accelerometers, and delivers stimulation to one or more target nerves.

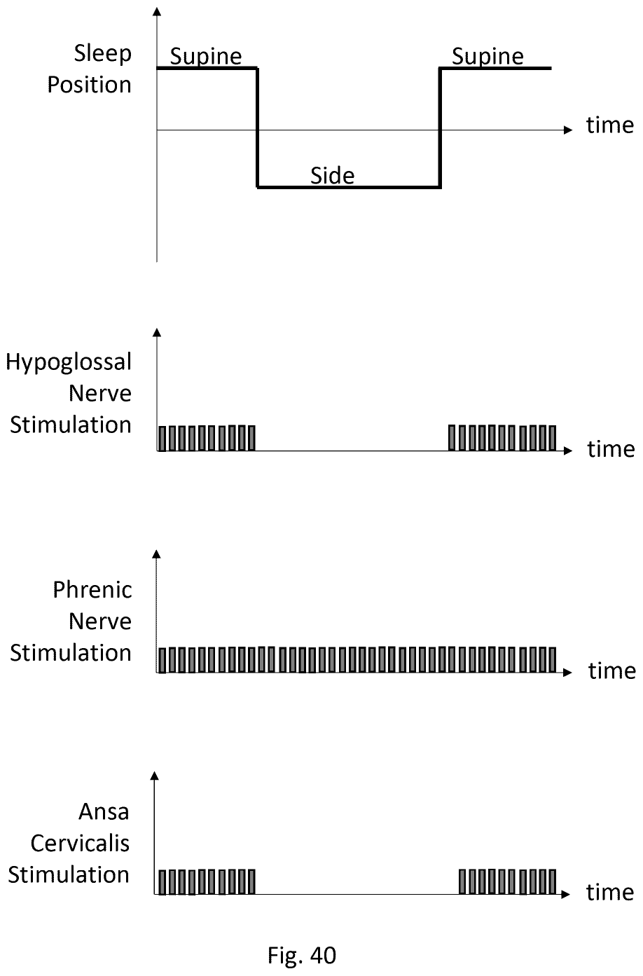

It is sometimes advantageous to stimulate multiple targets simultaneously since a patient might have an occlusion of the airway that may require the stimulation of different targets, or may require the stimulation of all targets to open the airway. Such a treatment option is further illustrated in FIG. 40.

When a patient is lying on his or her side, it is usually easier to open the airway, hence it is sufficient to stimulate only the phrenic nerve. An advantage of stimulating only the phrenic nerve (where additional nerves are possible to stimulate) is that stimulating all the nerves may: 1) be uncomfortable for the patient; and/or 2) more quickly deplete batter power (e.g., use power unnecessarily). However, when the patient is in the supine position, airway occlusion may become worse, hence the stimulation of the hypoglossal nerve or even the ansa cervicalis may be needed as illustrated in FIG. 40.

According, in connection with certain example embodiments, sleep position of the patient can be determined. In some examples, sleep position may be determined from the linear accelerometers ACCEL_{X} and ACCEL_{Y} as shown in FIG. 09. In that case, the Sensor1 and Sensor2 of FIG. 39 can be the linear accelerometers ACCEL_{X} and ACCEL_{Y} of FIG. 09.

At times, it may be necessary to activate efferent neurons to trigger the contraction of the respiratory muscles, such as the stimulation of the phrenic nerve to cause the contraction of the diaphragm or the stimulation of the ansa cervicalis to cause the contraction of sternothyroid muscle or the stimulation of the hypoglossal muscle for the contraction of the hypoglossal muscle. At other times, it is beneficial to stimulate the afferent nerves to trigger the negative pressure reflex, instead or in addition to the stimulation of the efferent nerves. The selective stimulation of afferent nerves can be achieved by using the technique of nerve blocking that can be implemented with multipolar cuff electrodes.

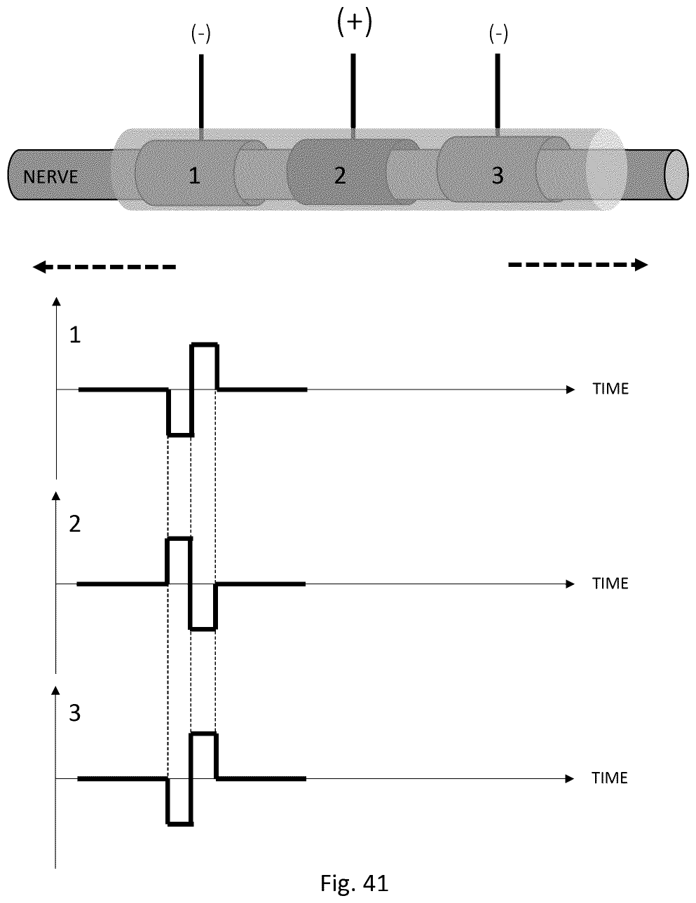

Multipolar cuff electrodes, as shown in FIG. 41, are designed to provide stimulation to the target tissue, usually a nerve, by multiple contacts. In the case that the multipolar cuff electrode contains three electrodes, stimulation is generated such that the two outer contacts carry an electrical potential that is opposite of the potential of the central contact. For example, when the central contact 2 is held at a positive potential, the outer contact 1 and 3 are held at a potential that is equal in amplitude, but opposite in sign, e.g. negative. This type of design concentrates the stimulation to the nerve that is being targeted and/or reduces the possibility of the stimulation of unintended tissues.

Multipolar cuff electrodes can be used for bi-directional or unidirectional stimulation. When bidirectional stimulation of a nerve is desired, electrical potentials as indicated in FIG. 41 are applied to the contacts of the multipolar cuff electrodes, where the inner contact 2 is held at a potential that is opposite of the potential of the potential of the outer contacts 1 and 3. Furthermore, the bidirectional stimulation of the nerve is generally initiated by the negative phase at the outer contacts and the positive phase at the inner contact, which is further illustrated in FIG. 41. Resulting action potential would travel in either direction, capturing both the afferent and efferent nerves.

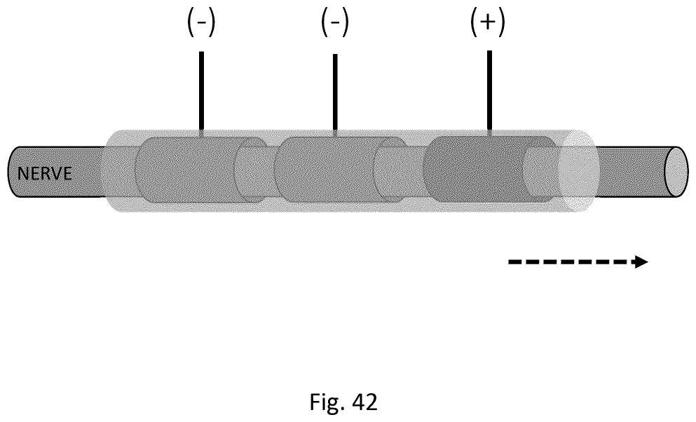

To generate a unidirectional nerve stimulation, the electrical pattern that is shown in FIG. 42 is used. For the generation of unidirectional stimulation, two contacts on the non-traveling direction of a three-contact electrode are kept at a negative potential while delivering a positive pulse to the contact on the traveling direction. This pattern allows the depolarization of the nerve on the travel direction while keeping the segments of the nerve on the non-travel direction hyperpolarized. Unidirectional stimulation of the nerve allows the selective capture of afferent or efferent nerves in a bundle that the cuff electrode surrounds.

### Additional Embodiments

Embodiment 1. A system to treat sleep disordered breathing, the system comprising: a nerve stimulator configured to selectively deliver stimulation energy to one or more of a plurality of nerves in a sleeping patient; at least one sensor configured to sense one or more physical aspects that are indicative of breaths taken by the patient and output one or more signals representative of characteristics of breathing by the sleeping patient; and a controller configured to: receive the one or more signals, select, based on the one or more signals, a first one or more of the plurality nerves; control the nerve stimulator or cause the nerve stimulator to be controlled to deliver the stimulation energy to the first one or more of the plurality of nerves; determine, based on the one or more signals, a change in the characteristics of the breathing of the sleeping patient; select, based on the one or more signals and the change in the characteristics of the breathing, a second one or more of the plurality of nerves; and control the nerve stimulator or cause the nerve stimulator to be controlled to deliver the stimulation energy to the second one or more of the plurality of nerves.

Embodiment 2. The system of Embodiment 1, wherein the plurality of nerves include two or more of: hypoglossal nerve, ansa nerve, phrenic nerve, and Vagus nerve.

Embodiment 3. The system of Embodiment 2, wherein the first one of the one or more of the plurality of nerves includes at least one of the hypoglossal nerve, the ansa nerve, the phrenic nerve and the Vagus nerve, and the second one of the one or more of the plurality of nerves includes at least one of the hypoglossal nerve, the ansa nerve, the phrenic nerve and the Vagus, wherein the second one includes one of the hypoglossal nerve, the ansa nerve, the phrenic nerve and the Vagus nerve that is not included in the first one.

Embodiment 4. The system of any of Embodiments 1 to 3, wherein the first one or more of the plurality of nerves includes afferent nerves and the second one or more of the plurality of nerves includes efferent nerves.

Embodiment 5. The system of Embodiment 4, wherein the stimulation energy that is delivered modulates one or more mechanoreflexes to restore airway patency and airflow.

Embodiment 6. The system of claim 4, wherein the stimulation energy excites motor nerves and respiratory muscles.

Embodiment 7. The system of any of Embodiments 1 to 6, wherein the stimulation rate applied to the first one or more of the plurality of nerves is a different rate than the stimulation rate applied to the second one or more of the plurality of nerves.

Embodiment 8. The system of any one of Embodiments 1 to 7, wherein the nerve stimulator includes electrodes each configured to be positioned proximate to a respective one of the plurality of nerves.

Embodiment 9. The system of any one of Embodiments 1 to 8, wherein the determination of the change in the characteristics of the breathing includes a determination that the sleeping patient has moved sleep positions.

Embodiment 10. The system of any one of Embodiments 1 to 9, wherein the controller controls the nerve stimulator or causes the nerve stimulator to be controlled to deliver the stimulation energy to the first and/or the second one or more of the plurality of nerves includes delivering the stimulation energy to excite both afferent and efferent nerves to cause opening of a breathing airway in the patient due to the simulation energy.

Embodiment 11. The system of any one of Embodiments 1 to 10, further comprising nerve block devices positioned proximate to each of the plurality of nerves, and the nerve stimulator is further configured to control the nerve block devices or cause the nerve block devices to block activation of nerves not included in the first one or more of the plurality of nerves while the first group is receiving the stimulation energy.

Embodiment 12. The system of any one of Embodiments 1 to 11, further comprising nerve block devices positioned proximate to each of the plurality of nerves, and the nerve stimulator is further configured to control the nerve block devices or cause the nerve block devices to block activation of nerves not included in the second one or more of the plurality of nerves while the second group is receiving the stimulation energy.

Embodiment 13. The system of any of Embodiments 1 to 12, wherein the controller is further configured to: determine, based on the signals, a respiratory cycle of the patient; determine, based on the signals and data indicating effects on the breathing of stimulation energy previously delivered to the nerve, a targeted period within the respiratory cycle; control the nerve stimulator or cause the nerve stimulator to be controlled to deliver the stimulation energy to the nerve synchronized with the targeted period, determine, based on the signals, whether the characteristics of the breathing indicate the breathing of the patient is inadequate; based on the determination, adjust one or more characteristics of the simulation energy delivered to the nerve.

Embodiment 14. The system of any of Embodiments 1 to 13, wherein the controller is further configured to: control the nerve stimulator or cause the nerve stimulator to deliver stimulation energy to the first and/or second one or more plurality of nerves at a stimulation rate synchronized with a natural breathing rate of the patient.

Embodiment 15. The system of Embodiment 14, wherein the natural breathing rate is determined while the patient is awake, and optionally the adjusted stimulation rate is in a range of 10 stimulations per minute slower than the natural breathing rate to 10 stimulations per minute faster than the natural breathing rate.

Embodiment 16. The system of any of Embodiments 1 to 15, wherein the controller is further configured to: control the nerve stimulator or cause the nerve stimulator to deliver stimulation energy in trains of pulses to the nerve and separate the trains by silent periods during which no significant stimulation energy is delivered to the nerve, adjust a length of each of the silent periods, based on a determination of the signals, and optionally the silent periods are each in a range of: 1 second to 60 seconds, 5 seconds to 45 seconds, 15 seconds to 60 seconds, or 60 seconds to five minutes.

Embodiment 17. The system of any of Embodiments 1 to 16, wherein the controller is further configured to: control the nerve stimulator or cause the nerve stimulator to deliver stimulation energy in trains of pulses to the nerve, and determine a phase in a respiratory cycle of the patient at which each train is delivered and/or a length of each of the trains.

Embodiment 18. The system according to any of Embodiments 1 to 17, wherein the one or more sensors are at least one of a: transthoracic impedance sensor, an accelerometer, a gyroscope, an auscultatory sensor, ultrasonic sensor, a pressure sensor, an optical sensor, a pulse oximeter, or a chemical sensor.

Embodiment 19. The system of any of Embodiments 1 to 18, wherein the controller is further configured to: initially control the nerve stimulator or cause the nerve stimulator to deliver the stimulation energy to the nerve at a stimulation rate synchronized with a natural breathing rate of the patient determined while the patient is awake, wherein the adjustment includes adjusting the stimulation rate, based on the determination, and optionally adjusting the stimulation rate within a range of 6 to 30 stimulations per minute.

Embodiment 20. The system of any of Embodiments 1 to 19, wherein the controller is further configured to deliver the stimulation energy to the nerve in pulse trains.

Embodiment 21. The system of Embodiment 20, wherein each pulse train is in a range of 0.2 to 2 seconds.

Embodiment 22. The system of Embodiments 20 or 21, wherein each pulse train includes energy pulses occurring at a frequency in a range of 10 Hz to 100 Hz.

Embodiment 23. The system of any of Embodiments 20 to 22, wherein a silent period is between each successive ones of the trains.

Embodiment 24. The system of Embodiments 23, wherein the silent period is in one of a range of: 1 second to 60 seconds, 5 seconds to 45 seconds, 15 seconds to 60 seconds, or 60 seconds to five minutes.

Embodiment 25. The system of any of Embodiments 13 to 24, wherein the targeted period is synchronized with an onset of inspiration in the respiratory cycle.

Embodiment 26. The system of any of Embodiments 13 to 25, wherein the targeted period is determined by applying a delay period to the onset of inspiration.

Embodiment 27. The system of Embodiment 26, wherein the delay period is before, coincides with or is after the onset of inspiration.

Embodiment 28. The system of any of Embodiments 13 to 27, wherein the targeted period occurs during an expiratory period of the respiratory cycle.

Embodiment 29. The system of any of Embodiments 13 to 27, wherein the targeted period occurs in a second half of the expiratory period.

Embodiment 30. The system of any of Embodiments 13 to 27, wherein the targeted period occurs in a first half of the expiratory period.

Embodiment 31. The system of any of Embodiments 13 to 27, wherein the targeted period occurs during a middle third of the expiratory period.

Embodiment 32. The system of any of Embodiments 1 to 31, wherein the breathing characteristics include one or more of the tidal volume, air flow, or airway patency.

Embodiment 51. A system to treat sleep disordered breathing, the system comprising: a nerve stimulator configured to deliver stimulation energy to a nerve in a sleeping patient; at least one sensor configured to sense one or more physical aspects that are indicative of a physiological condition of the patient, and output one or more signals indicative of the respiratory cycle of the patient; and a controller configured to: receive the one or more signals, determine a targeted period in the respiratory cycle, and control the nerve stimulator or cause the nerve stimulator to be controlled to deliver the stimulation energy to the nerve based on the targeted period.

Embodiment 52. The system according to Embodiment 51, wherein the controller causes or controls the nerve stimulator to deliver the stimulation energy synchronized with the target period.

Embodiment 53. The system of any of Embodiments 51 to 52, wherein the sensor is at least one of a: transthoracic impedance sensor, an accelerometer, a gyroscope, an auscultatory sensor, ultrasonic sensor, a pressure sensor, an optical sensor, a pulse oximeter, or a chemical sensor.

Embodiment 54. The system of any of Embodiments 51 to 53, wherein the nerve stimulation is configured to deliver the stimulation energy to the phrenic nerve.

Embodiment 55. The system of any of Embodiments 51 to 54, wherein the targeted period is synchronized with an onset of inspiration in the respiratory cycle.

Embodiment 56. The system of Embodiment 55, wherein the targeted period is determined by applying a delay period to the onset of inspiration.

Embodiment 57. The system of Embodiment 56, wherein the delay period is before, coincides with, or is after the onset of inspiration.

Embodiment 58. The system of any of Embodiments 51 to 57, wherein the targeted period occurs during an expiratory period of the respiratory cycle.

Embodiment 59. The system of Embodiment 58, wherein the targeted period occurs in a second half of the expiratory period.

Embodiment 60. The system of Embodiment 58, wherein the targeted period occurs in a first half of the expiratory period.

Embodiment 61. The system of Embodiment 58, wherein the targeted period occurs during a middle third of the expiratory period.

Embodiment 62. The system of any of Embodiments 51 to 61, wherein the nerve stimulator includes a lead providing power to a nerve stimulation electrode and the lead is integrated with the at least one sensor.

Embodiment 63. The system of any of Embodiments 51 to 62, wherein at least one sensor includes at least two electrodes configured to form a vector.

Embodiment 64. The system of any of Embodiments 51 to 63, wherein at least one sensor includes at least two electrodes configured to form a vector.

Embodiment 65. The system of any of Embodiments 51 to 64, wherein

the controlling the nerve stimulator includes delivering a train of electrical energy pulses to the nerve.

Embodiment 66. The system of Embodiment 65, wherein the train is

repeated during a sleep cycle of the patient.

Embodiment 67. The system of Embodiment 65 or 66, wherein the train is in a range of 0.2 to 2 seconds.

Embodiment 68. The system of any of Embodiments 65 to 67, wherein a silent period is between each successive one of the trains.

Embodiment 69. The system of Embodiment 68, wherein the silent period is in one of a range of: 1 second to 60 seconds, 5 seconds to 45 seconds, 15 seconds to 60 seconds, or 60 seconds to five minutes.

Embodiment 70. The system of any of Embodiments 65 to 69, wherein the electrical energy pulses in the train occur at a frequency of 10 Hz to 100 Hz.

Embodiment 71. The system of any of Embodiments 66 to 70, wherein the electrical energy pulses are biphasic.

Embodiment 72. The system of any of Embodiments 66 to 70, wherein the electrical energy pulses are monophasic.

Embodiment 73. The system of any of Embodiments 51 to 72, wherein the sleep disordered breathing is one or more of: obstructive sleep apnea, central sleep apnea, mixed sleep apnea or hypopnea.

Embodiment 74. The system of any of Embodiments 51 to 73, wherein the controller is further configured to: determine based on the one or more signals from the one or more sensors that breathing of the patient is insufficient; based on the determination, adjust the target period, and controlling the nerve stimulator to deliver the stimulation energy to the nerve during the adjusted targeted period.

Embodiment 75. The system of any of Embodiments 51 to 74, wherein the targeted period is predetermined before a sleep cycle of the patient.

Embodiment 76. The system of any of Embodiments 51 to 75, wherein the targeted period is determined during the sleep cycle of the patient.

Embodiment 77. The system according to any of Embodiments 51 to 76, wherein the controller is further configured to: receiving a command from a user interface communicatively connected to the controller indicative of the patient resting or sleeping, and/or receiving a command from a user interface communicatively connected to the controller indicative of the patient interrupting resting or sleeping, wherein the command is used by the controller to determine if the patient is asleep.

Embodiment 78. The system according to any Embodiments 1 to 77, wherein establishing whether the patient is resting or sleeping comprises: identifying a current time of the day, comparing the current time of the day with one or more pre-set time intervals, the one or more pre-set time intervals being stored in a memory communicatively connected with, or part of, the controller and being indicative of one or more periods in the day during which the patient is considered as resting or sleeping.

Embodiment 79. The system according to any of Embodiments 51 to 78, wherein the at least one sensor includes a circuit and a signal processor.

Embodiment 80. The system according to any of Embodiments 51 to 79, wherein the at least one sensor includes an acceleration sensor and a transthoracic impedance sensor.

Embodiment 81. The system according to Embodiment 80, wherein the at least one signal includes an output signal from the acceleration sensor and an output signal from the transthoracic impedance sensor.

### LIST OF ELEMENTS IDENTIFIED IN FIGURES

Patient 1
pharyngeal airway (pharynx, airway) 2
negative inspiratory pressure in the airway 3
positive pressure outside the airway 4
pharyngeal dilator muscles (e.g. genioglossus) 5
mandible 6
increase of lung volume 7
Soft pallet (vellum) 8
velopharynx or velopharyngeal space 9
CNS respiratory center 10
physiologic sensors 11
genioglossus 14
afferent limb of reflex 12
efferent limb of reflex 13
medulla 16
phasic activation of pharyngeal dilator muscles 20
pharyngeal patency 21
compromised pharyngeal anatomy 22
reflex reduction 24
sleep onset 23
reduced response to negative pressure 25
airway closure 26
hypoxia and hypocapnia 27
increase respiratory effort 28
arousal 29
phrenic nerve stimulation 30
augmented negative pressure 31
activation of afferent limb of NPR 32
restored activity of dilators 33
IPG 41
electrode system 42
diaphragm 43
phrenic nerve 44
lung 45
stimulation system 46
lead 47
airflow 50
respiratory effort 51
oxygen desaturations during apnea 52
control period when stimulation is turned off 52 54
periods when turned on 53, 55, 56
stimulation current level 55
increased current level 56
first stimulation burst 60
second stimulation burst 61
beginning of effort 62
Inspiration turns into expiration 63
cessation of effort 65
time delay 66
airway opens, beginning of inspiratory airflow 67
obstructed but not closed airflow 69
delay time 71
Inflection point 73
inspiration is terminated by the respiratory 74
exhales point 75.
Pulse period (frequency) 1110a, 111b
Pulse amplitude (voltage or power) 1120a, 1120b
Pulse train 1140a, 1140b
Pulse width 1130c, 1130c
Silent period between pulse trains 1145a, 1145b
transthoracic impedance signal 2302
Respiration waveform 3910
Implantable Stimulator 1600
Single lead for stimulation and transthoracic impedance sensing 1610
Cuff electrode of a single lead system 1620
Distal excitation electrode of a tripolar impedance measurement system in a single lead configuration 1630
Distal measurement electrode of a tripolar impedance measurement system in a single lead configuration 1640
Transthoracic impedance that is being measured with a single lead configuration 1640.

1. A system to treat sleep disordered breathing, the system comprising:
a nerve stimulator configured to deliver stimulation energy to a phrenic nerve in a sleeping patient;
at least one sensor configured to sense one or more physical aspects that are indicative of a physiological condition of the patient, and output one or more signals indicative of a respiratory cycle of the patient; and
a controller configured to:
receive the one or more signals,
determine, based on the one or more signals indicative of a respiratory cycle of the patient, a targeted period in the respiratory cycle, and
control the nerve stimulator or cause the nerve stimulator to be controlled to deliver the stimulation energy to the nerve based on the targeted period.

2. The system of claim 1, wherein the sensor is at least one of a:
transthoracic impedance sensor, an accelerometer, a gyroscope, an auscultatory sensor, ultrasonic sensor, a pressure sensor, an optical sensor, a pulse oximeter, or a chemical sensor.

3. The system of claim 1, wherein the at least one sensor includes an acceleration sensor and a transthoracic impedance sensor.

4. The system of claim 3, wherein the at least one signal includes an output signal from the acceleration sensor and an output signal from the transthoracic impedance sensor.

5. The system of claim 1, wherein the at least one sensor includes a 6-axis accelerometer that is configured to detect 3 transverse linear accelerations and 3 rotational accelerations, wherein the 6 signals are fused together for a chest acceleration signal,
wherein the one or more signals indicative of the respiratory cycle of the patient includes the chest acceleration signal.

6. The system of claim 1, wherein the at least one sensor includes a transthoracic impedance sensor.

7. The system of claim 6, wherein the transthoracic impedance sensor includes a bipolar configuration of electrodes.

8. The system of claim 6, wherein the transthoracic impedance sensor includes a tripolar configuration of electrodes.

9. The system of claim 1, wherein a common lead is used by the at least one sensor to sense the one or more physical aspects and the nerve stimulator to deliver stimulation energy to the phrenic nerve.

10. The system of claim 1, wherein a first sensing lead is used by the at least one sensor to sense the one or more physical aspects and a second lead is included with the nerve stimulator to deliver stimulation energy to the phrenic nerve, the first lead and second lead being separate from one another.

11. The system of claim 1, wherein the targeted period is synchronized based on a determined onset of inspiration in the respiratory cycle, wherein the targeted period is determined by applying a delay period to the determined onset of inspiration.

12. The system of claim 11, wherein the delay period is prior to onset of inspiration in the respiratory cycle of the patient.

13. The system of claim 1, wherein the controlling the nerve stimulator includes delivering a train of electrical energy pulses to the nerve.

14. The system of claim 13, wherein the train is in a range of 0.2 to 2 seconds.

15. The system of claim 13, wherein the electrical energy pulses in the train occur at a frequency of 10 Hz to 100 Hz.

16. The system of claim 13, wherein the electrical energy pulses are biphasic.

17. The system of claim 1, wherein the controller is further configured to:
determine based on the one or more signals from the one or more sensors that breathing of the patient is insufficient;
based on the determination, adjust the target period; and
control the nerve stimulator to deliver the stimulation energy to the nerve during the adjusted targeted period.

18. The system of claim 1, wherein the controller is further configured to:
receive a command from a user interface communicatively connected to the controller indicative of the patient resting or sleeping, and/or
receive a command from a user interface communicatively connected to the controller indicative of the patient interrupting resting or sleeping,
wherein the command is used by the controller to determine if the patient is asleep.

19. The system of claim 18, wherein establishing whether the patient is resting or sleeping comprises:
identifying a current time of day,
comparing the current time of the day with one or more pre-set time intervals, the one or more pre-set time intervals being stored in a memory communicatively connected with, or part of, the controller and being indicative of one or more periods in the day during which the patient is considered as resting or sleeping.

20. A method to treat sleep disordered breathing, the method comprising:
delivering, via a never stimulator, stimulation energy to a phrenic nerve in a sleeping patient;
obtaining, based on output from at least one sensor configured to sense one or more physical aspects that are indicative of a physiological condition of the patient, one or more signals indicative of a respiratory cycle of the patient;
determining, based on the one or more signals that are indicative of the respiratory cycle of the patient, a targeted period in the respiratory cycle, and controlling the nerve stimulator or causing the nerve stimulator to be controlled to deliver the stimulation energy to the nerve based on the targeted period.
